(19) United States
(12) Patent Application Publication
(10) Pub. No.: US 2010/0322880 A1
(43) Pub. Date: Dec. 23, 2010

(54) UVA FILTERS BASED ON ASCORBIC ACID DERIVATIVES

(75) Inventors: Thomas Rudolph, Darmstadt (DE); Philipp Buehle, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH

(21) Appl. No.: 12/866,281

(22) PCT Filed: Jan. 14, 2009
(86) PCT No.: PCT/EP09/00183

§ 371 Date: Aug. 5, 2010

(30) Foreign Application Priority Data
Feb. 6, 2008 (EP) 08002176.9

(51) Int. Cl. A61K 8/49; A61K 31/34; A61K 31/7048; C07H 17/04; C07D 307/02; A61K 8/19; A61K 8/33; A61Q 5/00; A61Q 19/00; A61Q 17/04; A61Q 3/02; A61Q 1/02

(52) U.S. Cl. 424/59; 514/474; 514/32; 536/4.1; 549/479; 424/70.9; 424/63; 424/70.1

(57) ABSTRACT

The invention relates to compounds of formula (I) wherein R¹, R², R³, R⁴ have the meaning cited in the claims, to methods for the production thereof, to agents containing said compounds and to their use for the functionalisation of matrices, in particular their use as skin and/or hair-binding UV filters.

The invention relates to the use of at least one UVA filter based on ascorbic acid derivatives, and to specific ascorbic acid derivatives and processes for the preparation thereof.

The functionalisation according to the invention of protein-like matrices, in particular the skin, hair and/or nails, is carried out by covalent anchoring or strong electrostatic interaction. This results in immobilisation of the desired active compounds, for example of UVA filters.

A preferred area of application of the use according to the invention is UVA protection. The human skin is subject to certain ageing processes, some of which are attributable to intrinsic processes (chronoageing) and some of which are attributable to exogenous factors (environmental, for example photoageing).

The exogenous factors include, in particular, sunlight or artificial radiation sources having a comparable spectrum, and compounds which are able to form due to the radiation, such as undefined reactive photoproducts, which may also be free radicals or ionic.

A multiplicity of organic and inorganic UVA filters and antioxidants which are able to absorb UVA radiation and scavenge free radicals is known. They are thus able to protect the human skin. These compounds catalyse the transformation of UV light into heat.

Owing to poor skin adhesion, however, the duration of protection is limited, in particular since conventional UVA filters can be washed off very easily, for example by sweat or water.

It is known, for example from WO 2006/018104, to derivatise UV filters in such a way that they can bond covalently to the stratum corneum of the epidermis via a reactive moiety and thus functionalise the skin with the UV filter. For effective bonding to proteins and amino acids in the outer layers of the skin, it is necessary for the corresponding UV filter derivatives to have the highest possible reactivity of their bonding-capable moieties.

There is therefore increasing demand for skin-tolerated compounds which are able to functionalise protein-containing matrices and can be incorporated in a suitable manner into cosmetic or pharmacological preparations.

Surprisingly, it has now been found that ascorbic acid derivatives, in particular ascorbic acid derivatives which are substituted in the 6- and/or 5-position by active-compound radicals, are highly suitable for the functionalisation of matrices. In addition, it has now surprisingly been found that hydrophobicisation of the derivatives enables their stability to be increased very considerably. For the purposes of this invention, improved stability means improved stability of the derivatives to oxidation and/or to hydrolysis and/or to heat and/or to electromagnetic radiation (for example UVA light).

This effect appears, in particular, if, in part B of the molecule, as described below, the two alkyl units A′ in NA′₂ of R¹⁰ or the alkyl unit A′ in OA′ of R¹⁰ each consist of at least 5 non-aromatic C atoms. In addition, this type of hydrophobicisation of the molecule enables the use concentration in the end product to be increased and consequently the efficacy to be considerably increased.

Preferred matrices here are skin, hair and/or nails, where the general principle can also be applied to the functionalisation of synthetic polymer matrices containing amino groups or thiol groups, isolated proteins or gelatine. The products formed by bonding to such matrices can also themselves be used as cosmetic active compounds for the preparation of cosmetic compositions. Both D- and also L-ascorbic acid, or mixtures thereof, can be derivatised in accordance with the invention.

The invention therefore relates firstly to the use of at least one UVA filter based on ascorbic acid derivatives.

It is known that ascorbic acid (vitamin C), often employed as natural anti-oxidant in the cosmetics or food industry, undergoes considerable losses due to vitamin C degradation depending on various parameters, such as oxygen, pH, metal-ion concentration (for example of iron or copper) or temperature. [H.-D. Belitz, W. Grosch, Lehrbuch der Lebensmittelchemie [Textbook of Food Chemistry], Springer-Verlag, 1987, 3rd Edition, p. 337.]

EP 0664290 describes derivatives of ascorbic acid in which the 2-position or also the 2- and 6-position is esterified by cinnamic acid. These ascorbic acid derivatives are used as antioxidants or, in accordance with EP 104631, as NO donors.

EP 0917871 describes ascorbic acid derivatives whose hydroxyl group in the 4-position is substituted by C₁-C₆-alkoxycarbonyl and whose hydroxyl groups in the 5- and/or 6-position are substituted by C₁-C₂₀-acyl or C₁-C₆-alkoxycarbonyl, where the acyl chains are branched, unbranched, saturated or (poly)unsaturated, i.e. based on fatty acids. Aromatic systems are excluded. These compounds are also used as antioxidants

EP 1527777 describes ascorbic acid derivatives in which at least one hydroxyl group of the ascorbic acid has been esterified by means of a benzoic acid, preferably a gallic acid. The compounds are described, inter alia, as inhibitors of tyrosine activity or as inhibitors of melanin synthesis. The use according to the invention for the functionalisation of matrices is not mentioned or even suggested.

G. Tschank et al, Biochem. J. 1994, 300, 75-79, describe the compounds O⁶-(2-acetoxybenzoyl) L-ascorbate and O⁵O⁶-bis(2-acetoxybenzoyl) L-ascorbate. These compounds are capable of supporting the activity of the enzyme prolyl-4-hydroxylase, but the disubstituted compound has lower affinity to the enzyme.

Particularly suitable for the use according to the invention is at least one ascorbic acid derivative of the formula

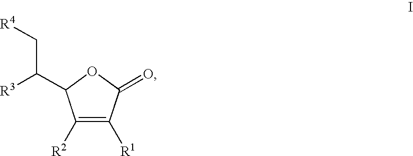

where
R¹ or R² each, independently of one another, denote hydroxyl, —O-alkyl, —OC(O)-alkyl, —OPO₃M or O-glycosyl,
alkyl denotes C₁-C₂₀-alkyl,
M denotes an alkali or alkaline-earth metal cation or H,
R³ or R⁴ each, independently of one another, denote hydroxyl or a radical B and
B denotes the radical of a UVA filter,
with the proviso that at least one of the radicals R³ or R⁴ stands for a radical B and that R¹⁰ in the formula II of the radical B indicated below stands for C₁-C₂₀ alkyl, NA′₂ or OA′, where A′ denotes branched or linear C₅-C₂₀ alkyl.
C₁-C₂₀-alkyl denotes an alkyl group having 1 to 20 C atoms, for example methyl, ethyl, propyl, n-butyl, tert-butyl, n-pentyl, n-hexyl, 2-ethylhexyl, n-dodecyl or lauryl.
C₅-C₂₀-alkyl, in relation to the radical B for A′ in NA′₂ or OA′, denotes an alkyl group having 5 to 20 C atoms, for example n-pentyl, n-hexyl, n-octyl, 2-ethylhexyl, tert-butylmethyl, 2,5-dimethyl hexyl, 1,3,5-trimethylheptyl, n-dodecyl, 8-ethyldodecyl, 6-propylundecyl, 5-ethyl-3-methyldecyl, 4-hexyldecyl, 2-pentylnonyl or n-lauryl.

Alkoxy radicals which come into consideration for R¹ or R² are those whose alkyl group contains 1 to 20 C atoms, preferably 1 to 6 C atoms, particularly preferably 1 to 4 C atoms. Examples of alkoxy groups are methoxy, ethoxy, propoxy, isopropoxy, butoxy, isobutoxy or tent-butoxy.

The group —OPO₃M is preferably the —OPO₃H group, but it is also possible to employ salts of the formula I, where M in formula I corresponds to an alkali metal cation, for example of Na or K, or an alkaline-earth metal cation, for example of Mg or Ca.

The bonding of a carbohydrate in position 2 or 3 of the ascorbic acid, referred to as O-glycosyl in formula I, can take place, for example, for monosaccharides, such as ribose, arabinose xylose, lyxose, allose, altrose, glucose, mannose, gulose, idose, galactose, talose, ribulose, xylulose, psicose, fructose, sorbose or tagatose. This list covers both isomers, i.e. in each case the D or L forms.

Preference is given to the use of glucose, galactose or fructose, very particularly preferably glucose.

In principle, however, disaccharides, such as saccharose (or also known as sucrose), lactose, trehalose, maltose, cellobiose, gentiobiose or melibiose, are also suitable. This list includes both the α and also the β forms.

From the group of the disaccharides, preference is given to the use of saccharose or lactose, particularly preferably saccharose.

Preferably, the radical R¹ in formula I denotes hydroxyl and R² denotes —O-alkyl, —OC(O)-alkyl, —OPO₃M or O-glycosyl, as described above, where alkyl preferably denotes alkyl having 1 to 6 C atoms.

Preferably, the radical R² in formula I denotes hydroxyl and R¹ denotes —O-alkyl, —OC(O)-alkyl, —OPO₃M or O-glycosyl, as described above, where alkyl preferably denotes alkyl having 1 to 6 C atoms.

Particularly preferably, both radicals R² and R¹ are hydroxyl.

In a preferred embodiment, the radical R³ is hydroxyl and R⁴ corresponds to a radical B, as described above and below.

However, it is also possible in accordance with the invention to employ mixtures of ascorbic acid derivatives of the formula I in which the radical B stands both for R³ and R⁴, and also for R³ or R⁴.

The radical B, as described in greater detail above and below, is bonded to position 5 and/or 6 of the formula I via an ester function. The radical B is particularly preferably bonded via a carbonyloxy function.

The radicals R¹ and R² of the ascorbic acid derivatives of the formula I are selected in such a way that, on application to the matrix, in particular the skin, hair and/or nails, or also on application to isolated proteins or gelatine, bonds form to reactive groups of the matrix, such as amino and/or thiol groups. The bonding reaction is simplified if the ascorbic acid derivative of the formula I is activated by degradation through oxidation of the hydroxyl groups R¹ and/or R². The hydroxyl groups R¹ and/or R² can also form from an ascorbic acid derivative of the formula I where R¹ and/or R² ≠H by hydrolysis on application to the matrix.

The theory of further functionalisation of the matrix is described below via bonding activation by ascorbic acid degradation, although functionalisation of the matrices is not intended to be tied to this theory.

In Scheme 1, the ascorbic acid derivative decomposes to give xylosone and/or 4-desoxyperitosone, where, in the formulae of Scheme 1, R³ denotes OH or a radical B and R⁴ denotes a radical B, as described above:

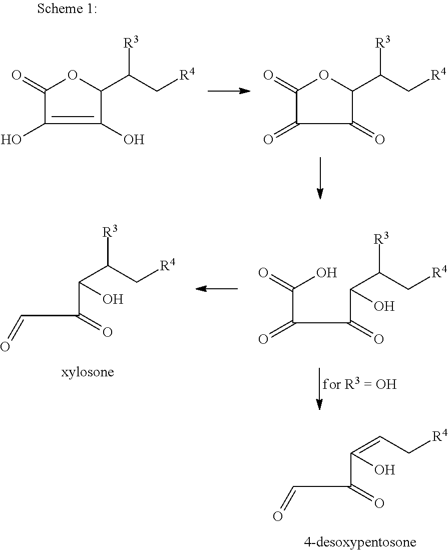

The reactive dicarbonyl compounds xylosone and 4-desoxypentosone are able to react with proteins and amino acids in the sense of a Maillard reaction. This step corresponds to integration of the active-compound-carrying radicals R³ and/or R⁴ into the matrix. The matrix is therefore functionalised corresponding to the active-compound radical,

Compared with, for example, non-bonding UV filters, this mechanism has two additional advantages, namely the antioxidative (degradation) reaction of the ascorbic acid skeleton and, where appropriate, a tanning reaction analogous to the Maillard reaction (self-tanning agent component).

In a variant of the invention, it is particularly preferred for the radical B in formula I to be a substituent which absorbs UVA radiation, for example a cosmetic UVA filter, preferably of the formula II,

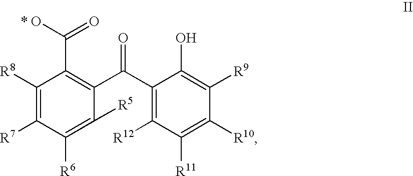

where
R⁵ to R⁹ and R¹¹ to R¹² each, independently of one another, denote H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl and
A is alkyl having 1 to 20 C atoms,
n is an integer from 1 to 25,
X is the counterion to the cations [NHA₂]⁺ and [NA₃]⁺ or to the anion [SO₃]⁻ and
Y and Z are each, independently of one another, -ascorbyl, hydroxyl, —O-2-ethylhexyl, —O-hexyl, —OA or —NH—C(CH₃)₃, and
R¹⁰ stands for A, NA′₂ or OA′, where A′ denotes branched or linear C₅-C₂₀ alkyl, particularly preferably having at least five linked C atoms. The hydrophobicity of the molecule as a whole can be controlled through the choice of suitable substituents A or A′ of substituent R¹⁰ of the radical B.

However, it is also possible for partial charges in the molecule to be self-balanced, i.e. for compounds of the formula I to be in the form of a zwitterionic structure.

Depending on the pH, these compounds may carry partial charges, as shown below for the compound (R)-2-((R)-3,4-dihydroxy-5-oxo-2,5-dihydrofuran-2-yl)-2-hydroxy-ethyl 2-(4-dihexylamino-2-hydroxybenzoyl)benzoate:

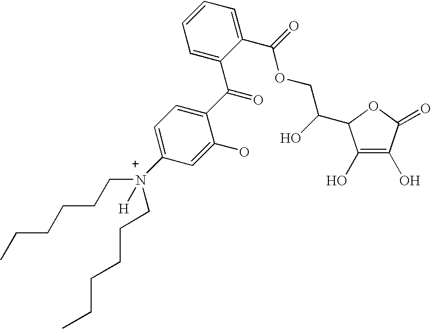

as ammonium compound,

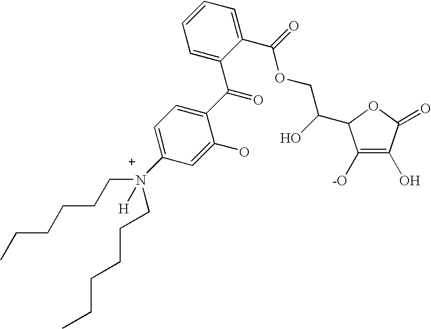

as zwitterionic compound or

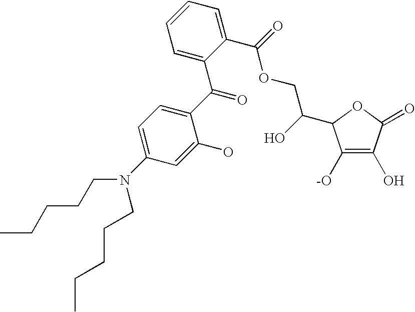

as ascorbate.

Compounds of the formula I can also be used in accordance with the invention as salts, i.e. at least one hydroxyl group of the ascorbic acid skeleton is in deprotonated form, and the charge is balanced by a countercation, for example an alkali or alkaline-earth metal cation.

Further preferred combinations are disclosed in the claims.

Particularly preferred embodiments of the compounds of the formula I can be seen in the moiety II for the radical B, as described above:

R¹⁰ of the radical B is preferably NA′₂ or ON, particularly preferably NA′₂, where A′ denotes branched or linear C₅-C₂₀ alkyl.

The compounds of the formula I, as described above, can generally be prepared by methods known per se to the person skilled in the art from the literature. The reaction conditions for esterifications are standard prior art, and the choice of suitable reaction conditions is part of the standard expert knowledge of the person skilled in the art of synthesis.

The invention furthermore relates to compounds of the formula I

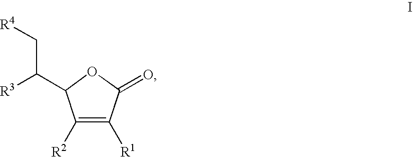

where
R¹ or R² is each, independently of one another, hydroxyl, —O-alkyl, —OC(O)-alkyl, —OPO₃M or O-glycosyl,
alkyl is C₁-C₂₀-alkyl,
M is an alkali or alkaline-earth metal cation or H,
R³ or R⁴ is each, independently of one another, hydroxyl or a radical B and
B is a radical of the formula II

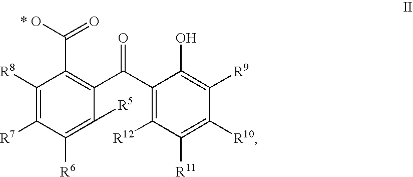

where
R⁵ to R⁹ and R¹¹ to R¹² each, independently of one another, denote H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl and
A is alkyl having 1 to 20 C atoms,
n is an integer from 1 to 25,
X is the counterion to the cations [NHA₂]⁺ and [NA₃]⁺ or to the anion [SO₃]⁻ and
Y and Z are each, independently of one another, -ascorbyl, hydroxyl, —O-2-ethylhexyl, —O-hexyl, —OA or —NH—C(CH₃)₃, and R¹⁰ stands for A, NA′₂ or OA′, where A′ denotes branched or linear C₅-C₂₀-alkyl and with the proviso that at least one of the radicals R³ or R⁴ stands for the radical B.

In a variant of the invention, preference is given to compounds of the formula I if R² in formula I denotes hydroxyl.

In a variant of the invention, preference is given to compounds of the formula I if R¹ in formula I denotes hydroxyl.

In a variant of the invention, preference is given to compounds of the formula I if R⁵ to R⁹, R¹¹ and R¹² in formula II denote H.

In a variant of the invention, particular preference is given to compounds of the formula I if R¹⁹ in formula II is NA′₂.

The invention likewise relates to a process for the preparation of compounds of the formula I, as described above, characterised in that

a) a compound of the formula III

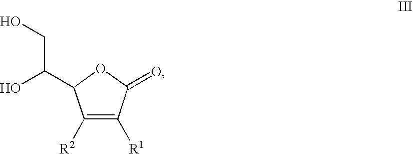

in which
R¹ or R² has one of the meanings indicated above for the formula I or indicated as preferred,
is reacted directly with a compound of the formula IV

B-M IV

in which B has one of the meanings described above, and
M denotes an alkali metal cation or alkaline-earth metal cation or H, or
b) the hydroxyl groups of the compound of the formula III, as described above, are protected to give a compound of the formula V

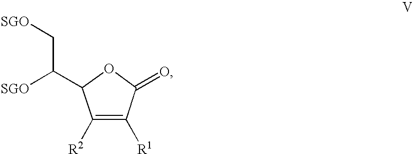

in which
R¹ or R² has one of the meanings described above for the formula I and SG denotes a protecting group,
in a subsequent step the radicals R¹ and/or R², if these are hydroxyl groups, are protected by a second protecting group which can be cleaved off again under different reaction conditions to the protecting group SG, the protecting groups SG of the compounds of the formula V are cleaved off again, and the resultant compound is reacted with a compound of the formula IV

B-M IV,

where B has a meaning described above for the formula I, and
M denotes an alkali metal cation or alkaline-earth metal cation or H, and the radicals R¹ and/or R² are subsequently deprotected to give the hydroxyl group, and these hydroxyl groups are optionally converted into another radical R¹ or R²≠OH, as described above.

The direct esterification of the compounds of the formula III using compounds of the formula IV, if these are bonded via a carbonyloxy function, is carried out in the presence of concentrated sulfuric acid and preferably under inert-gas conditions. The mixture of the components is advantageously prepared at temperatures <5° C. The actual reaction temperature is between 10 and 60° C., preferably between 15 and 30° C. The reaction is particularly preferably carried out at room temperature.

Some of the starting materials of the formulae III and IV are commercially available, for example ascorbic acid, ascorbic acid phosphate, sodium and magnesium ascorbyl phosphate, ascorbic acid glucoside, 2-(4-dihexylamino-2-hydroxybenzoyl)benzoic acid or 2-(4-dipentylamino-2-hydroxy-benzoyl)benzoic acid, or can be synthesised by methods which are described, for example, in the standard works, such as Houben-Weyl, Methoden der organischen Chemie [Methods of Organic Chemistry], Georg-Thieme-Verlag, Stuttgart, to be precise under reaction conditions which are known and suitable for the said reactions. Use can also be made here of variants known per se which are not mentioned here in greater detail.

In the case of direct esterification, a mixture of ascorbic acid C-6 esters and ascorbic acid C-5 esters forms as a consequence of the syn thesis, where the ascorbic acid C-6 ester generally predominates. Ascorbic acid C-6 esters are compounds of the formula I in which R⁴ denotes B. Ascorbic acid C-5 esters are compounds of the formula I in which R³ denotes B. These mixtures can of course be separated by methods which are known to the person skilled in the art.

The alternative preparation of the compounds according to the invention is essentially based on protecting-group chemistry of the hydroxyl groups of the compounds of the formula III, as defined above, in order that the esterification can take place specifically in position 5 and/or 6 of the ascorbic acid skeleton. However, the esterification using compounds of the formula IV can also be carried out without prior protecting-group chemistry, where the reaction conditions are adequately known to the person skilled in the art.

The protecting groups are generally selected to be different from one another so that they can be cleaved off selectively (cf. in this respect: T. W. Greene, P. G. M. Wuts, Protective Groups in Organic Chemistry, 2nd Edn., Wiley, New York 1991 or P. J. Kocienski, Protecting Groups, 1st Edn., Georg Thieme Verlag, Stuttgart-New-York, 1994, H. Kunz, H. Waldmann in Comprehensive Organic Syn thesis, Vol. 6 (eds. B. M. Trost, I. Fleming, E. Winterfeldt), Pergamon, Oxford, 1991, pp. 631-701).

The expression “hydroxyl-protecting group” is likewise known in general terms and relates to groups which are suitable for protecting a hydroxyl group against chemical reactions. Typical of such groups are unsubstituted or substituted aryl, aralkyl, aroyl or acyl groups, furthermore also alkyl groups, alkyl-, aryl- or aralkylsilyl groups or O,O- or O,S-acetals. The nature and size of the hydroxyl-protecting groups is not crucial since they are removed again after the desired chemical reaction or reaction sequence; preference is given to groups having 1-20, in particular 1-10, C atoms. Examples of hydroxyl-protecting groups are, inter glia, aralkyl groups, such as benzyl, 4-methoxybenzyl or 2,4-dimethoxybenzyl, aroyl groups, such as benzoyl or p-nitrobenzoyl, acyl groups, such as acetyl or pivaloyl, p-toluenesulfonyl, alkyl groups, such as methyl or tert-butyl, but also allyl, alkyl-silyl groups, such as trimethylsilyl (TMS), triisopropylsilyl (TIPS), tert-butyl-dimethylsilyl (TBS) or triethylsilyl, trimethylsilylethyl, aralkylsilyl groups, such as tert-butyldiphenylsilyl (TBDPS), cyclic acetals, such as isopropylidene, cyclopentylidene, cyclohexylidene, benzylidene, p-methoxybenzylidene or o,p-dimethoxybenzylidene acetal, acyclic acetals, such as tetrahydropyranyl (Thp), methoxymethyl (MOM), methoxyethoxymethyl (MEM), benzyloxymethyl (BOM) or methylthiomethyl (MTM). Particularly preferred hydroxyl-protecting groups are benzyl, acetyl, tert-butyl or TBS.

The starting material employed for the synthesis is preferably ascorbic acid whose hydroxyl groups in the 5- and 6-position are protected by protecting groups SG, as described above, by known methods to give compounds of the formula V. A cyclic protecting group which simultaneously effectively protects both positions 5 and 6 is advantageously selected. Examples of compounds of the formula V are accordingly 5,6-isopropylidene, cyclopentylidene, cyclohexylidene, benzylidene, p-methoxybenzylidene or o,p-dimethoxybenzylidene ascorbate. 5,6-Isopropylidene ascorbate is preferably employed.

The hydroxyl groups in positions 2 and 3 are subsequently protected with the aid of protecting groups, as described above, where an aralkyl group or an alkylsilyl group is advantageously selected, particularly preferably an aralkyl group, for example the benzyl group.

After liberation of the hydroxyl groups protected in the first step, a reaction is carried out with a compound of the formula IV, where B and M have a meaning described above.

If a reaction is carried out with a compound of the formula IV in which M=H and B corresponds to the sub-formula II, the coupling reaction is preferably carried out in the presence of a dehydrating agent, for example a carbodiimide, such as dicyclohexylcarbodiimide (DCC), N-(3-dimethylaminopropyl)-N′-ethylcarbodiimide hydrochloride (EDC) or diisopropylcarbodiimide (DIC), furthermore, for example, propanephosphonic anhydride (cf. Angew. Chem. 1980, 92, 129), diphenylphosphoryl azide or 2-ethoxy-N-ethoxycarbonyl-1,2-dihydroquinoline, in an inert solvent, for example a halogenated hydrocarbon, such as dichloromethane, an ether, such as tetrahydrofuran or dioxane, an amide, such as DMF or dimethylacetamide, a nitrile, such as acetonitrile, in dimethyl sulfoxide or in the presence of these solvents, at temperatures between about −10 and 40, preferably between 0 and 30°. Depending on the conditions used, the reaction time is between a few minutes and several days.

Instead of compounds of the formula IV, as defined above, it is also possible to employ derivatives of the formula IV, preferably a pre-activated carboxylic acid or a carboxylic acid halide, a symmetrical or mixed anhydride or an active ester. Radicals of this type for activation of the carboxyl group in typical acylation reactions are described in the literature (for example in the standard works, such as Houben-Weyl, Methoden der organischen Chemie [Methods of Organic Chemistry], Georg-Thieme-Verlag, Stuttgart).

Activated esters are advantageously formed in situ, for example by addition of HOBt (1-hydroxybenzotriazole) or N-hydroxysuccinimide.

The reaction is generally carried out in an inert solvent, on use of a halide of the formula IV in the presence of an acid-binding agent, preferably an organic base, such as triethylamine, dimethylaniline, pyridine, dimethyl-aminopyridine or quinoline.

The addition of an alkali or alkaline-earth metal hydroxide, carbonate or bicarbonate or of another salt of a weak acid of the alkali or alkaline-earth metals, preferably of potassium, sodium, calcium or caesium, may also be favourable.

After coupling and thus introduction of the active-compound-providing radical into the skeleton of ascorbic acid is complete, the hydroxyl groups in positions 2 and 3 are deprotected, giving compounds of the formula I in which R¹ and R² denote hydroxyl. The conversion of these hydroxyl groups, if desired, into other radicals R¹ and R², as defined above, is carried out by standard methods.

The ascorbic acid derivatives described are capable of bonding to textiles or textile fibres and thus developing their action in each case depending on the radical B, for example UVA protection.

The ascorbic acid derivatives according to the invention in which the radical B is a substituent which absorbs UVA radiation and has a conjugated π-electron system of at least 4 π electrons, containing the moiety of the formula II, also have, for example, anti-ageing effects and have the advantages for the skin that are derived from ascorbic acid, i.e. they serve, for example, for skin regeneration and cause wrinkle reduction of (light)-aged skin, they furthermore increase, for example, the skin relief density or strengthen, for example, the dermis-epidermis bond (papilla index). They protect the skin against UV-induced damage or they have, for example, a skin-bleaching action. They have, for example, an antibacterial action, i.e. they can reduce sweat odour or improve the skin appearance in the case of skin impurities and/or acne.

The ascorbic acid derivatives according to the invention in which the radical B is a substituent which absorbs UVA radiation and has a conjugated π-electron system of at least 4 π electrons, containing the moiety of the formula II, are capable of bonding to hair and can thus suppress hair damage caused by UVA light or by oxidation, in particular with respect to colour and morphology. For example, protection can thus be provided against bleaching of the hair.

The ascorbic acid derivatives according to the invention in which the radical B is a substituent which absorbs UVA radiation and has a conjugated π-electron system of at least 4 π electrons, containing the moiety of the formula II, are capable of bonding not only to nitrogen-containing, but also to sulfur-containing hair functionalities, such as, for example, to thiolic groups. Owing to the good reduction properties of the compounds, the ascorbic acid derivatives according to the invention can be employed, for example through controlled reduction of disulfide bridges, in hair-treatment products for defrizzing or for the formation of permanent waves.

EP 1728501 describes the use of UVA light-protection filters which are bonded to a polypeptide. Analogously to the teaching of EP 1728501, the ascorbic acid derivatives according to the invention in which the radical B is a substituent which absorbs UVA radiation and has a conjugated π-electron system of at least 4 π electrons, and conforms to the moiety of the formula II, can be combined with an amino acid, peptide or protein or bonded to an amino acid, peptide or protein before application.

Corresponding to the preferred use of the compounds according to the invention as skin- and/or hair-binding UV filters, the present invention furthermore relates to a composition, for example a cosmetic, dermatological or pharmaceutical preparation or composition, comprising at least one compound of the formula I

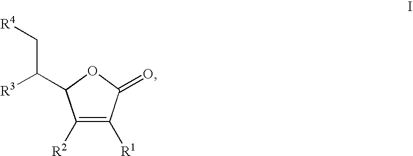

where
R¹ or R² each, independently of one another, denote hydroxyl, —O-alkyl, —OC(O)-alkyl, —OPO₃M or O-glycosyl, alkyl is C₁-C₂₀-alkyl,
M is an alkali or alkaline-earth metal cation or H,
R³ or R⁴ is each, independently of one another, hydroxyl or a radical B and
B is a substituent of the formula II,

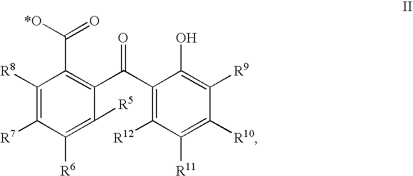

where
R⁵ to R⁹ and R¹¹ to R¹² each, independently of one another, denote H, —OH, —OA, -A, —NH₂, —NHA, —NA₂, —NH—(CH₂—CH₂—O)ₙ—H, —NH—(CH₂—CH₂—O)ₙ—H, —N[(CH₂—CH₂—O)ₙ—H]₂, —[NHA₂₁X, —[NA₃]X, —SO₃H, 4SO₃]X or 2H-benzotriazol-2-yl and
A is alkyl having 1 to 20 C atoms,
n is an integer from 1 to 25,
X is the counterion to the cations [NHA₂]⁺ and [NA₃]⁺ or to the anion [SO₃] and
Y and Z are each, independently of one another, -ascorbyl, hydroxyl, —O-2-ethylhexyl, —O-hexyl, —OA or —NH—C(CH₃)₃, and
R¹⁰ in the formula II stands for A, NA′₂ or OA′, where A′ denotes branched or linear C₅-C₂₀ alkyl, particularly preferably having at least five linked C atoms,
with the proviso that at least one of the radicals R³ or R⁴ stands for the radical B.

The number n stands for an integer from 1 to 25, preferably for an integer of 1, 2, 3, 4 or 5.

X describes the counterion for the cations [NHA₂] and [NA₃]⁴, where A has the meanings indicated above, preferably Cl⁻, Br⁻, I⁻ or [SO₄]²⁻ or the counterion of the anion [SO₃]⁻, preferably an ammonium ion or an alkali or alkaline-earth metal cation, such as Na⁺, Mg²⁺ or Ca²⁺.

However, it is also possible for partial charges to be compensated in the molecule itself, i.e. for compounds of the formula I to be in form of a zwitterionic structure.

Compounds of the formula I can also be used in accordance with the invention as salts, i.e. at least one hydroxyl group of the ascorbic acid skeleton is in deprotonated form and the charge is compensated by a countercation, for example an alkali or alkaline-earth metal cation.

Advantages of the compounds or preparations according to the invention here, besides the absorbent action as UVA filters; are, in particular, the antioxidant action, which develops on functionalisation of the matrix through decomposition of the ascorbic acid skeleton, if appropriate the self-tanning action which arises from the Maillard reaction, and in particular the functionalisation of the matrix which, in the case of the active-compound radical B of the sub-formula II in the compounds of the formula I, corresponds to immobilisation of the active compound and thus, for example, an immobilised UVA-protection action.

However, the compounds according to the invention also have an antioxidant action for structural reasons.

For the purposes of the present invention, the term preparation or formulation is used synonymously.

The compositions here are usually either preparations which can be applied topically, for example cosmetic, pharmaceutical or dermatological formulations. In this case, the preparations comprise a cosmetically, pharmaceutically or dermatologically suitable vehicle and, depending on the desired property profile, optionally further suitable ingredients. The topical preparations are preferably employed as cosmetic or dermatological preparation, particularly preferably as cosmetic preparation.

The compounds of the formula I are typically employed in accordance with the invention in amounts of 0.01 to 20% by weight, preferably in amounts of 0.05% by weight to 10% by weight. The person skilled in the art is presented with absolutely no difficulties here in selecting the amounts correspondingly depending on the intended action of the preparation.

The compositions according to the invention preferably comprise as little oxygen as possible, i.e. the compositions should be prepared under inert-gas conditions. It is furthermore advantageous to keep the water content low. It is furthermore advantageous to limit the presence of (heavy) metal ions since these, as is known, can destabilise antioxidants. Thus, the compositions according to the invention may comprise, for example, complexing agents. During preparation and also storage, the substances according to the invention, and the compositions comprising the substances according to the invention, should be protected against UV radiation, light and heat. If the compositions comprise water, the pH should preferably be set in the acidic range. All measures in this respect are known to the person skilled in the art.

However, the protective action against oxidative stress or against the action of free radicals can be improved further if the compositions or compositions according to the invention comprise one or more further antioxidants, where the person skilled in the art is presented with absolutely no difficulties in selecting antioxidants which act suitably quickly or in a time-delayed manner.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents, (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylidene-glucitol, carnosine, butylhydroxytoluene, butylhydroxyanisole, nordihydro-guaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the general formula A or B

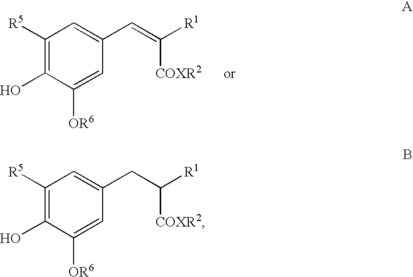

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes linear or branched alkyl having 1 to 8 C atoms or linear or branched alkoxy having 1 to 8 C atoms and
R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST liquid) and/or bis(2-ethylhexyl) 2-(4-hydroxy-3,5-dimethoxybenzyl)malonate (for example RonaCare® AP).
Mixtures of antioxidants are likewise suitable for use in the compositions or preparations according to the invention. Known and commercial mixtures are, for example, mixtures comprising, as active compounds, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in such compositions with compounds of the formula I in ratios in the range from 1000:1 to 1:1000, preferably in amounts of 100:1 to 1:100.
The compositions or compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably retinol, nicotinamide, vitamin A palmitate, vitamin C and derivatives thereof, DL-α-tocopherol, tocopherol E acetate, nicotinic acid, pantothenic acid and biotin, very particularly preferably retinol or nicotinamide. Vitamins are usually employed here with compounds of the formula I in ratios in the range from 1000:1 to 1:1000, preferably in amounts of 100:1 to 1:100.

Compositions or preparations which are particularly preferred in accordance with the invention also comprise pure UV filters in addition to the compounds of the formula I.

In principle, all UV filters are suitable for combination with the compounds of the formula I according to the invention. Particular preference is given to UV filters whose physiological acceptability has already been demonstrated. These UV filters are generally incorporated into cosmetic formulations in an amount of 0.5 to 20 percent by weight, preferably 1-15% by weight.

Both for UVA and UVB filters, there are many proven substances known from the specialist literature, for example

benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyl-dibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

Conceivable inorganic UV filters are those from the group of the titanium dioxides, such as, for example, coated titanium dioxide (for example Eusolex®T-2000, Eusolex®T-AQUA, Eusolex®T-AVO), zinc oxides (for example Sachtotec®), iron oxides or also cerium oxides. These inorganic UV filters are generally incorporated into cosmetic compositions in an amount of 0.5 to 20 percent by weight, preferably 2-10%.

Combination of one or more compounds of the formula I with further UV filters enables the protective action against damaging effects of UV radiation to be optimised. Broadband protection systems thereby arise, which can also be supplemented by addition of inorganic UV filters.

All the said UV filters can also be employed in encapsulated form. In particular, it is advantageous to employ organic UVA filters in encapsulated form. In detail, the following advantages arise:
The hydrophilicity of the capsule wall can be set independently of the solubility of the UV filter. Thus, for example, it is also possible to incorporate hydrophobic UV filters into purely aqueous preparations. In addition, the oily impression on application of the preparation comprising hydrophobic UV filters, which is frequently regarded as unpleasant, is suppressed.

Encapsulation of these filters or compounds which impair the photostability of these filters, such as, for example, cinnamic acid derivatives, enables the photostability of the entire preparation to be increased.

Skin penetration by organic UV filters and the associated potential for irritaion on direct application to the human skin is repeatedly being discussed in the literature. The encapsulation of the corresponding substances which is proposed here suppresses this effect.

In general, encapsulation of individual UV filters or other ingredients enables preparation problems caused by the interaction of individual preparation constituents with one another, such as crystallisation processes, precipitation and agglomerate formation, to be avoided since the interaction is suppressed.

It is therefore preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active compound (UVA filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules particularly preferably to be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subjectmatter of the present application.

The capsules are preferably present in compositions or preparations according to the invention in amounts which ensure that the encapsulated UVA filters are present in the preparation in the above-indicated amounts.

The compositions or preparations according to the invention may in addition comprise further anti-ageing active compounds, anti-cellulite active compounds or conventional skin-protecting or skin-care active compounds. Skin-protecting or skin-care active compounds can in principle be any active compounds known to the person skilled in the art.

Particularly preferred anti-ageing active compounds are pyrimidinecarboxylic acids, aryl oximes, bioflavonoids, bioflavonoid-containing extracts, chromones or retinoids.

Pyrimidinecarboxylic acids occur in halophilic microorganisms and play a role in osmoregulation of these organisms (E. A. Galinski et al., Eur, J. Biochem., 149 (1985) pages 135-139). Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid and derivatives thereof. These compounds stabilise enzymes and other biomolecules in aqueous solutions and organic solvents. They furthermore stabilise, in particular, enzymes against denaturing conditions, such as salts, extreme pH values, surfactants, urea, guanidinium chloride and other compounds.

Ectoin and ectoin derivatives, such as hydroxyectoin, can advantageously be used in medicaments. In particular, hydroxyectoin can be employed for the preparation of a medicament for the treatment of skin diseases. Other areas of application of hydroxyectoin and other ectoin derivatives are typically in areas in which, for example, trehalose is used as additive. Thus, ectoin derivatives, such as hydroxyectoin, can be used as protectant in dried yeast and bacteria cells. Pharmaceutical products, such as non-glycosylated, pharmaceutical active peptides and proteins, for example t-PA, can also be protected with ectoin or its derivatives.

Of the cosmetic applications, particular mention should be made of the use of ectoin and ectoin derivatives for the care of aged, dry or irritated skin. Thus, European patent application EP-A-0 671 161 describes, in particular, that ectoin and hydroxyectoin are employed in cosmetic preparations, such as powders, soaps, surfactant-containing cleansing products, lipsticks, rouge, make-up, care creams and sunscreen preparations.

Preference is given here to the use of a pyrimidinecarboxylic acid of the following formula

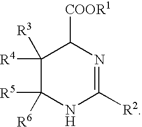

in which R¹ is a radical H or C₁₋₈-alkyl, R² is a radical H or C₁₋₄-alkyl, and R³, R⁴, R⁵ and R⁶ are each, independently of one another, a radical from the group H, OH, NH₂ and C₁₋₄-alkyl. Preference is given to the use of pyrimidinecarboxylic acids in which R² is a methyl or ethyl group and R¹ or R⁶ and R⁶ are H. Particular preference is given to the use of the pyrimidinecarboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid). The preparations according to the invention preferably comprise pyrimidinecarboxylic acids of this type in amounts of up to 15% by weight. The pyrimidinecarboxylic acids are preferably employed here in ratios of 100:1 to 1:100 with respect to the compounds of the formula I, with ratios in the range 1:10 to 10:1 being particularly preferred.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16123. Preparations which comprise 2-hydroxy-5-methyllaurophenone oxime are accordingly suitable for the treatment of skin diseases which are associated with inflammation. The preparations here preferably comprise 0.01 to 10% by weight of the aryl oxime, it being particularly preferred for the preparation to comprise 0.05 to 5% by weight of aryl oxime.

Known bioflavonoids are, for example, troxerutin, tiliroside, □-glucosylrutin, rutin or isoquercetin, where the said choice is not intended to have a restrictive effect.

Known anti-ageing substances are also chromones, as described, for example, in EP 1508327, or retinoids, for example retinol (vitamin A), retinoic acid, retinaldehyde or also synthetically modified compounds of vitamin A.

The chromones and retinoids described are simultaneously also effective anti-cellulite active compounds. A likewise known anti-cellulite active compound is caffeine.

The compositions may include, comprise or essentially consist of the said necessary or optional constituents or restrictions. All compounds or components which can be used in the compositions or preparations are either known and commercially available or can be synthesised by known processes.

One or more compounds of the formula I can be incorporated into cosmetic or dermatological preparations in a conventional manner. Suitable preparations are those for external use, for example in the form of a cream, lotion, gel or as a solution which can be sprayed onto the skin.

Examples which may be mentioned of application forms of the preparations according to the invention are: solutions, suspensions, emulsions, PIT emulsions, pastes, ointments, gels, creams, lotions, powders, soaps, surfactant-containing cleansing preparations, oils, aerosols and sprays. Examples of other application forms are sticks, shampoos and shower preparations. Any desired customary vehicles, auxiliaries and, if desired, further active compounds may be added to the preparation.

Preferred auxiliaries originate from the group of the preservatives, stabilisers, solubilisers, colorants, odour improvers.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, dimethyl capramide, dimethyl isosorbide, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

In a preferred application, the ascorbic acid derivatives according to the invention are, as described, converted into an application-suitable formulation just before use. For example, the substance is dissolved in a vehicle, as described above, and applied directly to skin or preferably to hair. Particularly suitable vehicles in this sense are Arlasolve DMI (Dimethylisosorbide), Butylene Glycol, Finsolv® PG-22 (Dipropylene Glycol Dibenzoate) or Pelemol® BIP (Butylphthalimide Isopropylphthalimide).

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, mascara, eyeliner, eye shadow, rouge, powder make-up, emulsion make-up and wax make-up, and sunscreen, pre-sun and after-sun preparations.

The preferred preparation forms according to the invention include, in particular, emulsions.

Emulsions according to the invention are advantageous and comprise, for example, the said fats, oils, waxes and other lipids, as well as water and an emulsifier, as usually used for a preparation of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic lipids, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and hydrocarbon waxes, silicone oils, dialkyl ethers, the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18, C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate, of cyclomethicone and 2-ethylhexyl isostearate.

The aqueous phase of the preparations according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other lipids, as well as water and an emulsifier, as usually used for a formulation of this type.

In a preferred embodiment, the preparations according to the invention comprise hydrophilic surfactants.

The hydrophilic surfactants are preferably selected from the group of the alkylglucosides, acyl lactylates, betaines and coconut amphoacetates.

The alkylglucosides are themselves advantageously selected from the group of the alkylglucosides which are distinguished by the structural formula

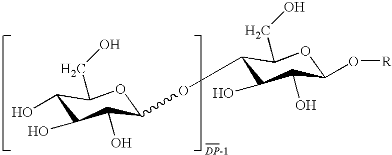

where R represents a branched or unbranched alkyl radical having 4 to 24 carbon atoms, and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = Σ \frac{{p}_{i}}{100} \cdot i$$

in which p₁, p₂, p₃ . . . pᵢ represent the proportion of mono-, di-, tri-1-fold glucosylated products in percent by weight. Advantageous in accordance with the invention is the selection of products having degrees of glucosylation of 1-2, particularly advantageously of 1.1 to 1.5, very particularly advantageously of 1.2-1.4, in particular of 1.3.

The value DP takes into account the fact that alkylglucosides generally, as a consequence of their preparation, represent mixtures of mono- and oligo-glucosides. A relatively high content of monoglucosides, typically in the order of 40-70% by weight, is advantageous in accordance with the invention.

Alkylglycosides which are particularly advantageously used in accordance with the invention are selected from the group octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and auxiliaries or mixtures which are distinguished by an effective content of the active compounds used in accordance with the invention, for example Plantaren® 1200 (Henkel KGaA), Oramix® NS 10 (Seppic).

The acyllactylates are themselves advantageously selected from the group of the substances which are distinguished by the structural formula

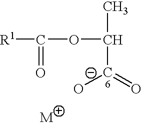

where R¹ denotes a branched or unbranched alkyl radical having 1 to 30 carbon atoms and M⁺ is selected from the group of the alkali metal ions and the group of ammonium ions which are substituted by one or more alkyl and/or one or more hydroxyalkyl radicals, or corresponds to half an equivalent of an alkaline-earth metal ion.

For example, sodium isostearyl lactylate, for example the product Pathionic® ISL from the American Ingredients Company, is advantageous.

The betaines are advantageously selected from the group of the substances which are distinguished by the structural formula

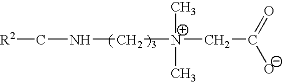

where R² denotes a branched or unbranched alkyl radical having 1 to 30 carbon atoms.

R² particularly advantageously denotes a branched or unbranched alkyl radical having 6 to 12 carbon atoms.

For example, capramidopropylbetaine, for example the product Tego® Betain 810 from Th. Goldschmidt AG, is advantageous.

A coconut amphoacetate which is advantageous in accordance with the invention is, for example, sodium coconut amphoacetate, as available under the name Miranol Ultra C32 from Miranol Chemical Corp.

The preparations according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological preparations according to the invention are applied to the skin and/or the hair in an adequate amount in the usual manner for cosmetics.

Cosmetic and dermatological preparations according to the invention can exist in various forms. Thus, they can be, for example, a solution, a water-free preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoin in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

Co-emulsifiers which are advantageous in accordance with the invention are, for example, O/W emulsifiers, principally from the group of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17)isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15)cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate,

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20)glyceryl oleate, polyethylene glycol (20) glyceryl isostearate, polyethylene glycol (18) glyceryl oleate/cocoate.

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Optional W/O emulsifiers, but ones which may nevertheless be advantageously employed in accordance with the invention are the following:

fatty alcohols having 8 to 30 carbon atoms, monoglycerol esters of saturated and/or unsaturated, branched and/or unbranched alkanecarboxylic acids having a chain length of 8 to 24, in particular 12-18, C atoms, diglycerol esters of saturated and/or unsaturated, branched and/or unbranched alkanecarboxylic acids having a chain length of 8 to 24, in particular 12-18, C atoms, monoglycerol ethers of saturated and/or unsaturated, branched and/or unbranched alcohols having a chain length of 8 to 24, in particular 12-18, C atoms, diglycerol ethers of saturated and/or unsaturated, branched and/or unbranched alcohols having a chain length of 8 to 24, in particular 12-18, C atoms, propylene glycol esters of saturated and/or unsaturated, branched and/or unbranched alkanecarboxylic acids having a chain length of 8 to 24, in particular 12-18, C atoms, and sorbitan esters of saturated and/or unsaturated, branched and/or unbranched alkanecarboxylic acids having a chain length of 8 to 24, in particular 12-18, C atoms.

Particularly advantageous W/0 emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG 30 dipolyhydroxystearate.

The compositions or preparations described are particularly suitable for protecting human skin against UV radiation, ageing processes and against oxidative stress, i.e. against damage caused by free radicals. In this connection, they are in the various administration forms usually used for this application. For example, the preparation may, in particular, be in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

The dyes used are preferably approved dyes which are listed in the Cosmetics Regulation, Annex 3, as positive list.

The preservatives used are preferably approved preservatives which are listed in the Cosmetics Regulation, Annex 6, as positive list or also anti-microbial pigments, as described, for example, in WO 2004/0092283 or WO 2004/091567.

Suitable preservatives are therefore also alkyl esters of p-hydroxybenzoic acid, hydantoin derivatives, propionate salts or a multiplicity of ammonium compounds.

Very particularly preferred preservatives are methylparaben, propylparaben, imidazolidinylurea, sodium dehydroxyacetate or benzyl alcohol. Preservatives are employed in amounts between 0.5 and 2% by weight.

Emollients or softeners are often incorporated into cosmetic preparations. They are preferably employed in 0.5 to 50% by weight, preferably between 5 and 30% by weight, based on the composition as a whole. In general, softeners can be classified in classes, such as, for example, the category of the esters, fatty acids or fatty alcohols, polyols, hydrocarbons and oils containing at least one amide structural unit.

Representative oils containing at least one amide structural unit together with their synthesis are described, in particular, in EP 1044676 and EP 0928608. A compound which is particularly preferred indicated is isopropyl N-lauroylsarcosinate, which is commercially available from Ajinomoto under the product name Eldew SL-205.

Of the esters, mono- or diesters can be selected. Examples in this respect are dibutyl adipate, diethyl sebacate, diisopropyl dimerate or dioctyl succinate. Branched fatty acid esters are, for example, 2-ethylhexyl myristate, isopropyl stearate or isostearyl palmitate. Tribasic esters are, for example, triisopropyl trilinoleate or trilauryl citrate. Straight-chain fatty acid esters are, for example, lauryl palmitate, myristyl lactate, oleyl erucate or stearyl oleate. Preferred esters are Coco-Caprylate/Caprate (=INCI name, these are esters made from coconut fatty alcohols with saturated medium-chain fatty acids), propylene glycol myristyl ether acetate, diisopropyl adipate or cetyl octanoate.

Suitable fatty alcohols and acids are compounds which have 10 to 20 C atoms. Particularly preferred compounds are cetyl, myristyl, palmitic or stearic alcohol or acid.

Suitable polyols are linear or branched-chain alkylpolyhydroxyl compounds, for example propylene glycol, sorbitol or glycerol. However, it is also possible to employ polymeric polyols, for example polypropylene glycol or polyethylene glycol. Butylene glycol and propylene glycol are also particularly suitable compounds for enhancing the penetration capacity.

Examples of hydrocarbons as softeners are compounds which generally have 12 to 30 C atoms. Specific examples are arylalkyl benzoates, alkyl benzoates, mineral oils, Vaseline, squalenes or isoparaffins.

Further emollients or hydrophobicising agents are preferably C₁₂ to C₁₅ alkyl benzoates, dioctyl adipate, octyl stearate, octyldodecanol, hexyl taurate, octyldodecyl neopentanoate, cyclomethicone, dicaprylic ether, dimethicone, phenyltrimethicone, isopropyl myristate, caprylic/capric glyceride, propylene glycol dicaprylate/dicaprate or decyf oleate.

A further category of functional ingredients of cosmetic preparations in the sense of the invention are thickeners. Thickeners are generally employed in amounts between 0.1 to 20% by weight, preferably between 0.5 to 10% by weight, based on the total amount. Examples of these compounds are crosslinked polyacrylate materials, commercially available from B. F. Goodrich Company under the trade name Carbopol. It is also possible to use thickeners such as xanthan gum, carrageenan gum, gelatine gum, karaya gum, pectin gum or carob seed flour.

Under certain circumstances, it is possible for a compound to be both a thickener and also a softener. Examples thereof are silicone gums (kinematic viscosity>10 centistokes), esters, such as, for example, glycerol stearate, or cellulose derivatives, for example hydroxypropylcellulose.

The dispersant or solubiliser used can be an oil, wax or other lipid, a lower monoalcohol or lower polyol or mixtures thereof. Particularly preferred monoalcohols or polyols include ethanol, i-propanol, propylene glycol, glycerol and sorbitol.

A preferred embodiment of the invention is an emulsion in the form of a protective cream or milk which, in addition to the compound(s) of the formula I, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The preparation according to the invention or the composition may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other lipids.

If a preparation is formulated as an aerosol, the customary propellants, such as alkanes, fluoroalkanes and chlorofluoroalkanes, are generally used.

The cosmetic preparation may also be used to protect the hair against photochemical damage in order to prevent colour changes, bleaching or damage of a mechanical nature. In this case, a suitable formulation is in the form of a shampoo, lotion, gel or emulsion for rinsing out, the preparation in question being applied before or after shampooing, before or after colouring or bleaching or before or after permanent waving. It is also possible to select a preparation in the form of a lotion or gel for styling and treating the hair, in the form of a lotion or gel for brushing or blow-waving, in the form of a hair lacquer, permanent waving composition, colorant or bleach for the hair. Besides the compound(s) of the formula I, the preparation having light-protection properties may comprise various adjuvants used in this type of composition, such as surface-active agents, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The present invention furthermore relates to a process for the preparation of a composition, as described above, characterised in that at least one compound of the formula I is mixed with a vehicle and optionally with further active compounds or auxiliaries. The present invention also relates to a process for preparing a preparation which is characterised in that at least one compound of the formula I containing radicals as described above is mixed with a cosmetically, pharmaceutically or dermatologically suitable vehicle.

The preparations or compositions according to the invention can be prepared here with the aid of techniques which are well known to the person skilled in the art.

The mixing can result in dissolution, emulsification or dispersion of the compound of the formula I in the vehicle.

The invention is explained in greater detail below with reference to examples. The invention can be carried out throughout the range claimed and is not restricted to the examples given here.

### EXAMPLES

### List of Abbreviations Used

eq. equivalent
DCC dicyclohexylcarbodilmide
DMAP dimethylaminopyridine
DMSO dimethyl sulfoxide
EDO N-(3-dimethylaminopropyl)-N′-ethylcarbodiimide hydrochloride
EA ethyl acetate
EG ethylene glycol
sat. saturated
conc. concentrated
1 N HCl 1 N hydrochloric acid
i-PrOH isopropanol
soln. solution
MeCN acetonitrile
MTBE methyl tert-butyl ether
org. organic
RT room temperature
hr. hour
T temperature
THF tetrahydrofuran

### Example 1A

### Synthesis of (R)-2-((R)-3,4-dihydroxy-5-oxo-2,5-dihydrofuran-2-yl)-2-hydroxyethyl 2-(4-dihexylamino-2-hydroxybenzoyl)benzoate (or synonymously ascorbyl 2-(4-dihexylamino-2-hydroxybenzoyl)benzoate)

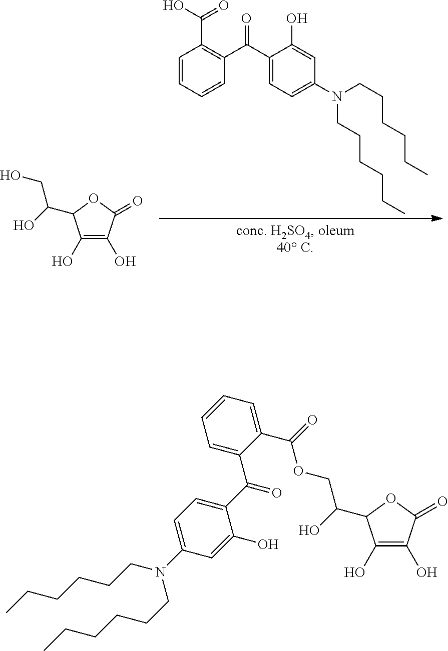

Vitamin C (24.8 g; 140.9 mmol, 4 eq.) is introduced in portions into 37.8 ml of conc. sulfuric acid in an argon-flushed apparatus. The internal temperature during this operation is kept below 5° C. by ice-cooling. 15 g of 2-(4-dihexylamino-2-hydroxybenzoyl)benzoic acid (35.2 mmol, 1 eq.) are subsequently introduced in portions, likewise at T<5° C. 15.6 ml of oleum are then added dropwise at T<15° C. After a reaction time of 6 hrs. at 40° C., the reaction solution is poured into 350 ml of ice-water. The mixture is extracted with 2×250 ml of MTBE. After drying over sodium sulfate, the solvent is removed in vacuo, giving the product as yellow foam (15.64 g; 76%).

In the reaction procedure, the compound (R)-1-((R)-3,4-dihydroxy-5-oxo-2,5-dihydrofuran-2-yl)-2-hydroxyethyl 2-(4-dihexylamino-2-hydroxybenzoyl)-benzoate also forms as a consequence of the synthesis.

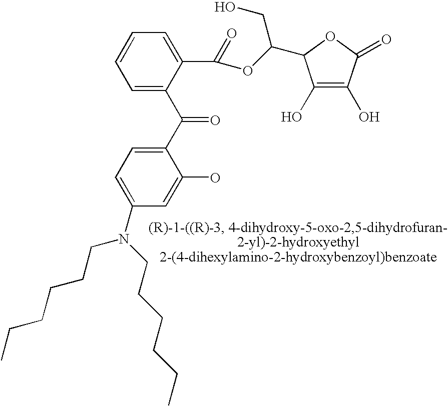

### Stability Data:

Cosmetic preparations having a constant content of in each case 1% of the compounds of the formula I according to the invention, for example (R)-2-((R)-3,4-dihydroxy-5-oxo-2,5-dihydrofuran-2-yl)-2-hydroxyethyl 2-(4-dihexylylamino-2-hydroxybenzoyl)benzoate, were prepared

The recovery of the UV filters is measured, measured after the preparation of the cosmetic formulation.

Human stratum corneum was incubated for 6 h as substrate with a 1% ethanolic solution of (R)-2-((R)-3,4-dihydroxy-5-oxo-2,5-dihydrofuran-2-yl)-2-hydroxyethyl 2-(4-dihexylylamino-2-hydroxybenzoyl)benzoate. The substrate was then washed with ethanol in order to remove unbound 4-dihexyl-aminobenzoic acid 6-O-ascorbate. The substrate is subsequently hydrolysed. The hydrolysate was measured photometrically. The absorption value achieved corresponded to the bound content of (R)-2-((R)-3,4-di hydroxy-5-oxo-2,5-dihydrofuran-2-yl)-2-hydroxyethyl 2-(4-dihexylylamino-2-hydroxybenzoyl)benzoate.

### Antioxidative Efficacy:

The basis for the determination of the antioxidative efficacy is the so-called DPPH test, as described in Bünger et. al. [Buenger, J., Ackermann, H., Jentzsch, A., Mehling, A., Pfizner, I., Reiffen, K-A., Schroeder, K.-R., and Wollenweber U., An interlaboratory comparison of methods used to assess antioxidant potentials, Int. J. Cosm. Sci., 28 (2006) 1-12]. The antioxidative efficacy of (R)-2-((R)-3,4-dihydroxy-5-oxo-2,5-dihydrofuran-2-yl)-2-hydroxyethyl 2-(4-dihexylylamino-2-hydroxybenzoyl)benzoate is determined in the DPPH test.

### Example 1B

### Synthesis of 2-(3,4-dihydroxy-5-oxo-2,5-dihydrofuran-2-yl)-2-hydroxyethyl 2-(4-dipentylamino-2-hydroxybenzoyl)benzoate

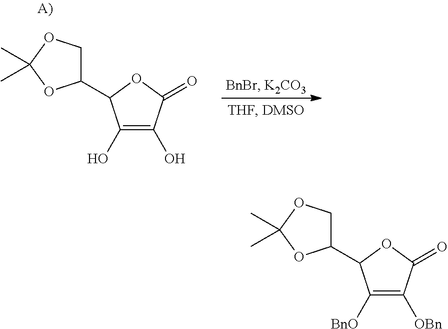

10 g of 5,6-isopropylidene ascorbate (46.3 mmol, 1 eq., available from Merck-Schuchardt: 8.18234) are dissolved in 30 ml of THF and 35 ml of DMSO, 19.2 g of potassium carbonate are added, and 13 ml of benzyl bromide are added dropwise (110 mmol, 2.4 eq.). After 3 hrs. at 50° C., the evolution of gas is complete. The solid is filtered off and extracted 3 times with 100 ml of ethyl acetate each time. The combined organic phases are dried over sodium sulfate, and the solvent is removed in vacuo. The product is obtained virtually quantitatively without further purification.

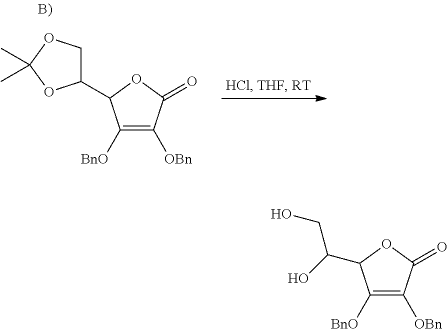

The di-benzyl-protected 5,6-isopropylidene ascorbate is dissolved in 65 ml of THF, and 30 ml of 2 N HCl are slowly added at room temperature. After 48 hrs., 150 ml of MTBE and solid sodium chloride are added to saturation, and the mixture is extracted. The organic phase is dried over sodium sulfate, and the solvent is removed in vacuo. The product is obtained virtually quantitatively without further purification.

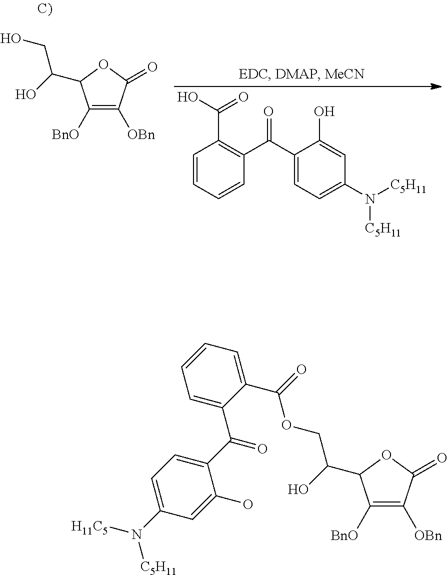

The di-benzyl-protected ascorbate (2.14 g; 6 mmol, 1 eq.) from step B) is dissolved in 11 ml of acetonitrile with DMAP (73 mg, 0.6 mmol, 0.1 eq.) and 1.88 g of 2-(4-dipentylamino-2-hydroxybenzoyl)benzoic acid (6 mmol, 1 eq.) in an argon-flushed flask. The EDC (1.7 g; 9 mmol, 1.5 eq.) is then added in portions at 0° C. The mixture is warmed to RT, and the solvent is removed in vacuo after 22 hrs. The residue is taken up with 50 ml of ethyl acetate and 50 ml of 1 N NaOH soln. and extracted. The org. phase is subsequently extracted with 2×50 ml of 1 N HCl and 1×50 ml of sat. NaCl soln. The organic phase is dried over sodium sulfate, and the solvent is removed in vacuo. The product is obtained after filtration through silica gel.

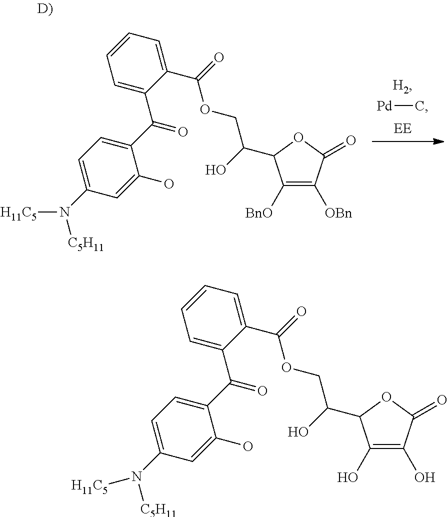

The starting material from step C) is dissolved in ethyl acetate and reduced using Pd/C catalyst under a hydrogen pressure of 1-5 bar. After filtration of the catalyst, the product is purified by filtration through silica gel.

### Example 1C

The following are prepared analogously to Example 1B:

2-(3,4-Dihydroxy-5-oxo-2,5-dihydrofuran-2-yl)-2-hydroxyethyl 2-(4-di[2-ethylhexyl]amino-2-hydroxybenzoyl)benzoate by reaction of the di-benzyl-protected ascorbate with 2-(4-di-[2-ethylhexyl]amino-2-hydroxybenzoyl)-benzoic acid and subsequent debenzylation:

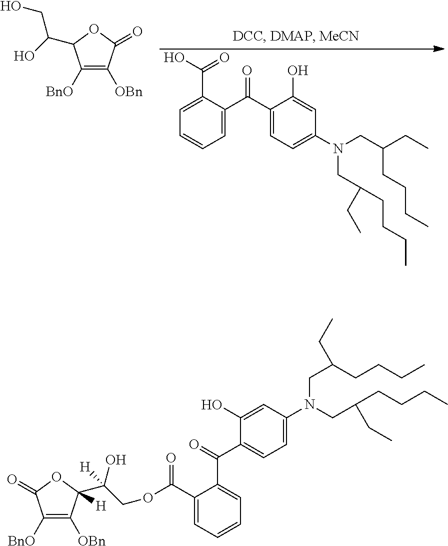

The di-benzyl-protected ascorbate (25 g; 70.2 mmol, 1 eq.) as from Example 1B step B) is dissolved in 125 ml of acetonitrile in an argon-flushed flask with DMAP (875 mg, 7 mmol, 0.1 eq.) and 50.7 g of 2-(4-di-(2-ethylhexyl)-amino-2-hydroxybenzoyl)benzoic acid (105.2 mmol, 1.5 eq.). DCC (21.7 g; 105.2 mmol, 1.5 eq.) is then added in portions at 0° C. The mixture is warmed to RT, and after 22 hrs. the solvent is removed in vacuo. The residue is taken up with 50 ml of ethyl acetate and 50 ml of 1 N NaOH soln. and extracted. The org. phase is subsequently extracted with 2×50 ml of saturated NH₄Cl and 1×50 ml of sat. NaCl soln. The organic phase is dried over sodium sulfate, and the solvent is removed in vacuo. The product is obtained as pale-yellow oil after filtration through silica gel.

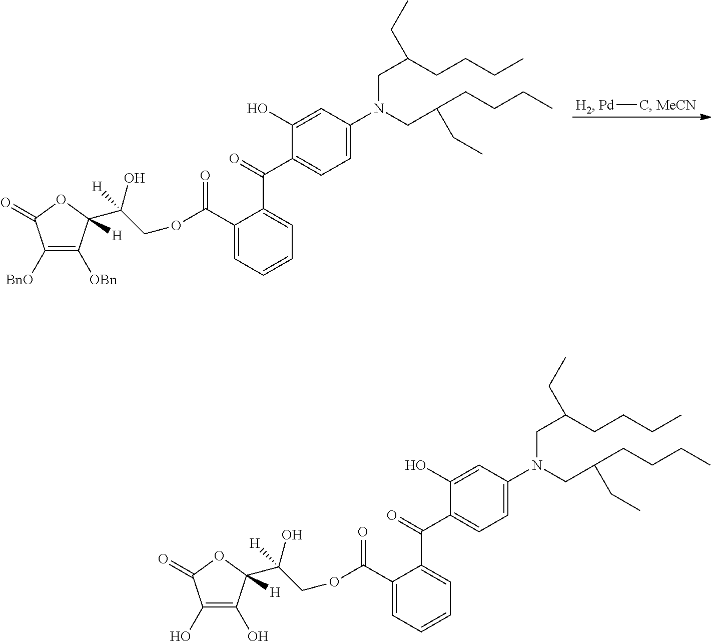

The product from step C) is dissolved in 250 ml of acetonitrile and reduced under a hydrogen pressure of 5 bar using 5 g of Pd/C catalyst. After filtration of the catalyst, the product is purified by filtration through silica gel, giving the product as a yellow oil.

### Example 1D

### 2-(3,4-Dihydroxy-5-oxo-2,5-dihydrofuran-2-yl)-2-hydroxyethyl 2-(4-di[n-octyl]amino-2-hydroxybenzoyl)benzoate by enzymatic esterification

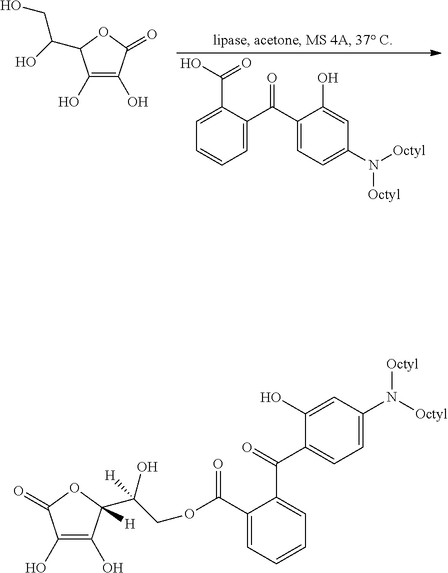

10 g of L-ascorbic acid (56.8 mmol; 1 eq.) are initially introduced in 190 ml of acetone with 10 g of 4 Å molecular sieve, 100 mg of lipase (for example Rhizomucor miehei, recombinant from Aspergillus oryzae) and then 72.9 g of 2-(4-di[n-octyl]amino-2-hydroxybenzoyl)benzoic acid (151.4 mmol, 2.67 eq.) are added. After a reaction time of 16 hrs at 37° C., the molecular sieve is filtered off, the mixture is cooled to room temperature, and the product is precipitated by slow addition of 100 ml of water. Drying at 60° C. in vacuo gives 2-(3,4-dihydroxy-5-oxo-2,5-dihydrofuran-2-yl)-2-hydroxyethyl 2-(4-di-[n-octyl]amino-2-hydroxy-benzoyl)benzoate as pale-yellow solid.

### Example 1E

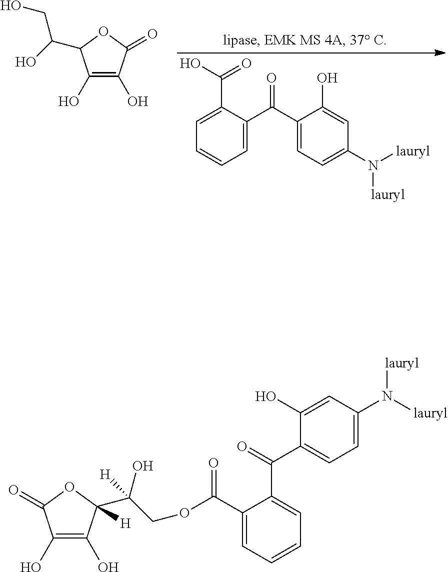

### 2-(3,4-Dihydroxy-5-oxo-2,5-dihydrofuran-2-yl)-2-hydroxyethyl 2-(4-di-lauryl-amino-2-hydroxybenzoyl)benzoate by enzymatic esterification

10 g of L-ascorbic acid (56.8 mmol; 1 eq.) are initially introduced in 250 ml of ethyl methyl ketone with 10 g of 4 Å molecular sieve, 150 mg of lipase (for example Candida cylindracea) and then 124.4 g of 2-(4-di-laurylamino-2-hydroxybenzoyl)benzoic acid (152 mmol, 2.67 eq.) are added. After a reaction time of 14 hrs at 37° C., the molecular sieve is filtered off, the mixture is cooled to room temperature, and the product is precipitated by slow addition of 125 ml of water and cooling to 5° C. Drying at 60° C. in vacuo gives 2-(3,4-dihydroxy-5-oxo-2,5-dihydrofuran-2-yl)-2-hydroxyethyl 2-(4-dilaurylamino-2-hydroxybenzoyl)benzoate as pale-yellow oil.

Formulations for cosmetic compositions are described by way of example below:

### Example 2

### W/O emulsion

| | 54-07-5-A | 54-07-5-B | 54-07-5-C | 54-07-5-D | 54-07-5-E |
|---|---|---|---|---|---|
| Cetyl PEG/PPG-10/1 | 3.00 | 3.00 | 3.00 | 3.00 | 3.00 |
| dimethicone | | | | | |
| (Abil EM 90) | | | | | |
| Polyglyceryl-4 | 1.50 | 1.50 | 1.50 | 1.50 | 1.50 |
| isostearate | | | | | |
| (Isolan GI 34) | | | | | |
| Butylphthalimide iso- | 5.00 | 5.00 | 5.00 | 5.00 | 5.00 |
| propylphthalimide | | | | | |
| (Pelemol ® BIP) | | | | | |
| Dimethyl isosorbide | 5.00 | 5.00 | 5.00 | 5.00 | 5.00 |
| (Arlasolve DMI) | | | | | |
| (R)-2-((R)--3,4- | 1.00 | | | 1.00 | 2.00 |
| Dihydroxy-5-oxo-2,5- | | | | | |
| dihydrofuran-2-yl)-2- | | | | | |
| hydroxyethyl 2-(4- | | | | | |
| dihexylylamino-2- | | | | | |
| hydroxybenzoyl)- | | | | | |
| benzoate | | | | | |
| Uvinul ® A Plus (DHHB) | | 0.84 | 0.84 | 1.00 | |
| Ascorbic acid | | | 0.37 | 1.00 | 3.00 |
| Mineral Oil | 8.00 | 8.00 | 8.00 | 8.00 | 8.00 |
| Ethylhexyl stearate | 5.00 | 5.00 | 5.00 | 5.00 | 5.00 |
| (Tegosoft ® OS) | | | | | |
| Cyclomethicone (and) | 5.00 | 5.00 | 5.00 | 5.00 | 5.00 |
| Aluminium/Magnesium | | | | | |
| Hydroxide Stearate | | | | | |
| (Gilugel SIL 5) | | | | | |
| Preservative | 1.00 | 1.00 | 1.00 | 1.00 | 1.00 |
| Water | to 100 | to 100 | to 100 | to 100 | to 100 |
| NaCl | 0.50 | 0.50 | 0.50 | 0.50 | 0.50 |
| EDTA | 0.10 | 0.10 | 0.10 | 0.10 | 0.10 |
| Citric acid q.s. | | | | | |

Preparation: Pelemol® BIP, Arlasolv DMI and emulsifiers are initially introduced. (R)-2-(R)-3,4-Dihydroxy-5-oxo-2,5-dihydrofuran-2-yl)-2-hydroxyethyl 2-(4-dihexylylamino-2-hydroxybenzoyl)benzoate

and Uvinul® A Plus are dissolved therein. The remaining constituents of the oil phase are added and mixed homogeneously. The water phase, adjusted to pH=4-5, is emulsified in with stirring. The mixture is subsequently homogenised. The emulsions can be prepared under gentle conditions at room temperature. (R)-2-((R)-3,4-Dihydroxy-5-oxo-2,5-dihydrofuran-2-yl)-2-hydroxyethyl 2-(4-dihexylylamino-2-hydroxy-benzoyl)benzoate can be stabilised by increasing the content of ascorbic acid. The preparation is ideally prepared with inertisation (exclusion of oxygen).

### Example 3

### Water-Resistant Sunscreen Spray

| A | | | |
|---|---|---|---|
| (R)-2-((R)--3,4-Dihydroxy-5-oxo- | 1.00 | 1.00 | 2.00 |
| 2,5-dihydrofuran-2-yl)-2- | | | |
| hydroxyethyl 2-(4-dihexylyl- | | | |
| amino-2-hydroxybenzoyl)- | | | |
| benzoate | | | |
| Diethylhexyl Syringylidene- | | 0.50 | |
| malonate, Caprylic/Capric | | | |
| Triglyceride | | | |
| (Oxynex ® ST liquid) | | | |
| RonaCare ® AP | | 2.00 | |
| Ascorbyl Palmitate | | | 1.00 |
| Cyprylic/capric Triglyceride | 7.00 | 7.00 | 7.00 |
| (Miglyol 812 N) | | | |
| Butylphthalimide isopropyl- | 10.00 | 10.00 | 10.00 |
| phthalimide | | | |
| (Pelemol ® BiP) | | | |
| C12-15 alkyl benzoate | 10.00 | 10.00 | 10.00 |
| (Tegosoft ® TN) | | | |
| Phenethyl benzoate | 5.00 | 5.00 | 5.00 |
| (X-Tend 226) | | | |
| RonaCare ® tocopherol acetate | 1.00 | 1.00 | 1.00 |
| B | | | |
| Cyclopentasiloxane | 43.80 | 41.30 | 41.80 |
| (Dow Corning 245) | | | |
| Phenyltrimethicone | 2.00 | 2.00 | 2.00 |
| (Dow Corning 556) | | | |
| Cyclopentasiloxane, | 20.00 | 20.00 | 20.00 |
| dimethiconol | | | |
| Dow Corning 1501 Fluid | | | |
| Perfume oil (q.s.) | 0.20 | 0.20 | 0.20 |

Preparation: the components of phase A are combined at room temperature and stirred until a clear solution is present. Phase B is subsequently mixed and added to phase B with stirring. Stirring is continued until finally the clear product is present. The stability of the substances according to the invention can be increased by addition of antioxidants, such as Oxynex® ST liquid, RonaCare® AP or ascorbyl palmitate.

### Example 4

### Pump Hairspray

| | | | |
|---|---|---|---|
| A | | | |
| (R)-2-((R)--3,4-Dihydroxy-5-oxo-2,5- | 1.00 | 2.00 | 4.00 |
| dihydrofuran-2-yl)-2-hydroxyethyl 2-(4- | | | |
| dihexylylamino-2-hydroxybenzoyl)- | | | |
| benzoate | | | |
| Ethanol 96% extra pure | to 100 | to 100 | to 100 |
| PVP/VA copolymer | 6.00 | 6.00 | 6.00 |
| PVP/VA W 735 | | | |
| B | | | |
| Diethylhexyl Syringylidenemalonate, | 0.06 | 0.25 | 0.50 |
| Caprylic/Capric Triglyceride | | | |
| (Oxynex ® ST Liquid) | | | |
| PEG-75 Lanolin BHT | 0.20 | 0.20 | 0.20 |
| (Solan E - low dioxane) | | | |
| Perfume | 0.10 | 0.10 | 0.10 |
| (Frag 280853 Green Activating) | | | |
| C | | | |
| Water, demineralised | 13.00 | 13.00 | 13.00 |
| Titriplex III | 0.10 | 0.10 | 0.10 |
| PEG-12 dimethicone | 0.50 | 0.50 | 0.50 |
| Dow Corning 193 Fluid | | | |
| 0.1% D&C Red No 33 (CI 17200) in | 0.20 | 0.20 | 0.20 |
| water | | | |
| PEG-40 Hydrogenated Castor Oil | 1.00 | 1.00 | 1.00 |
| (Cremophor RH 410) | | | |

Preparation: pre-dissolve phase A until a clear solution is present. Add phase B to phase A with stirring. Pre-mix phase C and add to the remainder, stir until a homogeneous mixture has formed.

### Example 5

### W/O Emulsions

| Emulsion | A | B | C | D | E | F |
|---|---|---|---|---|---|---|
| Polyglyceryl 2-dipolyhydroxy- | 3 | 5 | 3 | | | |
| stearate | | | | | | |
| PEG-30 dipolyhydroxystearate | | | 2 | 3 | 4 | 5 |
| Sodium starch octenylsuccinate | 0.5 | 0.4 | | 0.3 | | 1 |
| Glycine | 0.3 | 0.3 | 0.5 | 0.4 | | |
| Alcohol | | 5 | 2 | 5 | 4 | |
| Magnesium sulfate | 0.2 | 0.3 | 0.3 | 0.4 | 0.5 | 0.2 |
| C₁₂₋₁₅ alkyl benzoate | 5 | 3 | | | 5 | |
| C₁₂₋₁₃ alkyl tartrate | | 2 | | | | |
| Butylene glycol | 5 | | | | 3 | 3 |
| dicaprylate/dicaprate | | | | | | |
| Dicaprylyl ether | | | | | 2 | |
| Mineral oil | | 4 | | 6 | | 8 |
| Octyldodecanol | 2 | | | | | |
| Dicaprylic caprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene glckol | | | 1 | | 5 | 3 |
| Glycerine | 3 | 5 | 7 | 10 | 3 | 3 |
| C18-38 acid triglycerides | 0.5 | | 1 | | 1 | |
| Titanium dioxide | 5 | 6 | 4 | | | 4 |
| Zinc oxide | 5 | | | | | |
| Bisethylhexyloxyphenol- | | 3 | 3 | 2 | | |
| methoxyphenyltriazine | | | | | | |
| Ethylhexyltriazone | | 4.5 | 3 | | 3 | |
| (R)-2-((R)--3,4-Dihydroxy-5-oxo- | 2.0 | 0.5 | 1.0 | 1.0 | 3.0 | 1.5 |
| 2,5-dihydrofuran-2-yl)-2-hydroxy- | | | | | | |
| ethyl 2-(4-dihexylylamino-2- | | | | | | |
| hydroxybenzoyl)benzoate | | | | | | |
| Diethylhexylbutamidotriazone | | | 1.5 | 4 | | |
| Butylmethoxydibenzoylmethane | 2 | 3 | 4 | | 1 | 3 |
| Uvinul ® A Plus | | | | 4 | 2 | |
| Ethylhexyl methoxycinnamate | | | | | 7 | 5 |
| (R)-2-((R)--3,4-Dihydroxy-5-oxo- | 1.5 | 5.5 | | 8.0 | 4.5 | 7.5 |
| 2,5-dihydrofuran-2-yl)-2-hydroxy- | | | | | | |
| ethyl 2-(4-dihexylylamino-2- | | | | | | |
| hydroxybenzoyl)benzoate coupled | | | | | | |
| to gelatine | | | | | | |
| Benzotriazole coupled to | 4 | | 6 | | | |
| gelatine | | | | | | |
| Taurine | 0.1 | | | 0.5 | 0.2 | |
| Vitamin E acetate | 0.2 | 02 | | 0.3 | 0.1 | 0.5 |
| Na₂H₂EDTA | 0.1 | 0.1 | 0.2 | 0.2 | 0.2 | 0.5 |
| C8-C16 alkylpolyglycoside | 1 | | | | | |
| Perfume, preservatives | q.s. | q.s | q.s | q.s | qs. | qs. |
| Dyes, etc. | q.s. | q.s. | q.s. | q.s | q.s. | q.s. |
| Sodium hydroxide | q.s. | q.s. | q.s. | q.s | q.s. | q.s. |
| Water | to 100.0 | to 100.0 | to 100.0 | to 100.0 | to 100.0 | to 100.0 |

### Example 6

### Hair-Care Formulation

Content in g of component per 100 g of formulation

| Component | A | B | C | D | E | F |
|---|---|---|---|---|---|---|
| Disodium EDTA | 0.100 | 0.100 | 0.100 | 0.100 | 0.100 | 0.100 |
| Oxynex ® ST | 2.000 | 2.000 | 2.000 | 2.000 | 2.000 | 2.000 |
| (R)-2-((R)--3,4-Dihydroxy-5-oxo-2,5- | 0.10 | 0.25 | 0.50 | 1.50 | 2.00 | 4.00 |
| dihydrofuran-2-yl)-2-hydroxyethyl 2- | | | | | | |
| (4-dihexylylamino-2-hydroxy- | | | | | | |
| benzoyl)benzoate | | | | | | |
| Hexamidine diisethionate | 0.100 | 0 | 0 | 0 | 0 | 0 |
| Tetrahydrocurcumin | 0 | 0.500 | 0 | 0 | 0 | 0 |
| Glycyrrhetinic acid | 0 | 0 | 0.300 | 0 | 0 | 0 |
| Thiotaine ®¹ | 0 | 0 | 0 | 5.000 | 0 | 0 |
| N-undecylenoyl-L-phenylalanine | 0 | 0 | 0 | 0 | 1.000 | 0 |
| N-acetylglucosamine | 0 | 0 | 0 | 0 | 0 | 2.000 |
| Niacinamide | 5.000 | 5.000 | 5.000 | 5.000 | 5.000 | 5.000 |
| Citric acid | 0.015 | 0 | 0 | 0 | 0 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosylsarcosinate | 0 | 0 | 5.000 | 0 | 0 | 0 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl | 0.200 | 0.200 | 0.200 | 0.200 | 0.200 | 0.200 |
| alcohol | | | | | | |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerine | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 | 3.000 | 2.000 | 2.000 | 2.000 | 2.000 | 2.000 |
| isoparaffin + laureth-7 | | | | | | |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

### Example 7

### Hair-Care Formulation

Content in g of component per 100 g of formulation

| Component | G | H | I |
|---|---|---|---|
| Disodium EDTA | 0.100 | 0.100 | 0.100 |
| Oxynex ® ST | 2.000 | 2.000 | 2.000 |
| (R)-2-((R)--3,4-Dihydroxy-5-oxo-2,5- | 0.50 | 3.50 | 1.50 |
| dihydro-furan-2-yl)-2-hydroxyethyl 2-(4- | | | |
| dihexylyl-amino-2-hydroxybenzoyl)benzoate | | | |
| Cetylpyridinium chloride | 0.200 | 0 | 0 |
| Pitera ® | 0 | 10 | 0 |
| Ascorbyl glycoside | 0 | 0 | 2.000 |
| Niacinamide | 5.000 | 5.000 | 5.000 |
| Polyquaternium 37 | 0 | 0 | 0 |
| Isohexadecane | 3.000 | 3.000 | 3.000 |
| Isopropyl isostearate | 1.330 | 1.330 | 1.330 |
| Sucrose polycottonseedate | 0.670 | 0.670 | 0.670 |
| Polymethylsilsesquioxane | 0.250 | 0.250 | 0.250 |
| Cetearyl glucoside + cetearyl alcohol | 0.200 | 0.200 | 0.200 |
| Behenyl alcohol | 0.400 | 0.400 | 0.400 |
| Ethylparaben | 0.200 | 0.200 | 0.200 |
| Propylparaben | 0.100 | 0.100 | 0.100 |
| Cetyl alcohol | 0.320 | 0.320 | 0.320 |
| Stearyl alcohol | 0.480 | 0.480 | 0.480 |
| Tocopheryl acetate | 0.500 | 0.500 | 0.500 |
| PEG-100 stearate | 0.100 | 0.100 | 0.100 |
| Glycerine | 7.000 | 7.000 | 7.000 |
| Titanium dioxide | 0.604 | 0.604 | 0.604 |
| Polyacrylamide + C13-14 isoparaffin + | 2.000 | 2.000 | 2.000 |
| laureth-7 | | | |
| Panthenol | 1.000 | 1.000 | 1.000 |
| Benzyl alcohol | 0.400 | 0.400 | 0.400 |
| Dimethicone + dimethiconol | 2.000 | 2.000 | 2.000 |
| Water (to 100 g) | to 100 | to 100 | to 100 |
| TOTAL | 100 | 100 | 100 |

### Example 8

### O/W Emulsions

| Emulsion | A | B | C | D | E | F |
|---|---|---|---|---|---|---|
| Glyceryl stearate citrate | 2.5 | 2 | 3 | | | |
| Sorbitan stearate | 0.5 | | | 2 | 1.5 | 2 |
| Polyglyceryl-3 methylglycose | | | | 2.5 | 3 | 3 |
| distearate | | | | | | |
| Polyglyceryl-2 | | 0.8 | | | | 0.5 |
| dipolyhydroxystearate | | | | | | |
| Cetearyl alcohol | | | | 1 | | |
| Stearyl alcohol | 2 | | | | | 2 |
| Cetyl alcohol | | 1 | | | 3 | |
| Acrylates/C₁₀₋₃₀ alkyl acrylate | | 0.2 | | | 0.1 | |
| crosspolymer | | | | | | |
| Carbomer | | 0.2 | 0.3 | 0.2 | | |
| Xanthan gum | 0.4 | | 0.2 | 0.2 | 0.3 | 0.4 |
| C₁₂₋₁₅ alkyl benzoate | 5 | 3 | | | 5 | |
| C₁₂₋₁₃ alkyl tartrate | | 2 | | | | |
| Butylene glycol | 5 | | | | 3 | 3 |
| dicaprylat/dicaprat | | | | | | |
| Dicaprylyl ether | | | | | 2 | |
| Octyldodecanol | 2 | | | | | |
| Dicapryl caprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene gycol | | | 1 | | 5 | 3 |
| Glycerine | 3 | 5 | 7 | 10 | 3 | 3 |
| C18-C38 acid triglyceride | 0.5 | | 1 | | 1 | |
| Titanium dioxide | 5 | | | 2 | | |
| 2.2′-Methylenebis(6-(2H- | 2.5 | | | | | |
| benzotriazol-2-yl)-(1,1,3,3- | | | | | | |
| tetramethylbutyl)phenol) | | | | | | |
| 2.4,6-Tris(biphenyl)-1,3,5- | | 2 | | | | |
| triazine | | | | | | |
| Merocyanine coupled to | 6 | | 6 | | 10 | 3 |
| gelatine | | | | | | |
| Benzotriazole coupled to | | 5 | | 10 | | 3 |
| gelatine | | | | | | |
| C8-C16 alkylpolyglycoside | 1 | 0.6 | | | | |
| UVASorb ® K2A | | | 2 | | | |
| Uvinul ® A Plus | 2 | | | | | 1 |
| Homosalate | | 5 | | 1 | | |
| Phenylbenzimidazolesulfonic | | | 2 | | | 1 |
| acid | | | | | | |
| Benzophenone-3 | 2 | | | | 2 | |
| Octyl salicylate | 5 | 5 | | 2 | | |
| Octocrylene | 2 | | | | 3 | 1 |
| (R)-2-((R)--3,4-Dihydroxy-5- | 1.0 | 2.0 | 3.0 | 1.0 | 2.0 | 3.0 |
| oxo-2,5-dihydrofuran-2-yl)-2- | | | | | | |
| hydroxyethyl 2-(4-dihexylyl- | | | | | | |
| amino-2-hydroxybenzoyl)- | | | | | | |
| benzoate | | | | | | |
| Bisethylhexyloxyphenol- | | 3 | 2 | 1 | | |
| methoxyphenyltriazine | | | | | | |
| Parsol ® SLX | | | 3 | | | |
| Dihydroxy acetate | | | | | 4 | |
| Taurine | 0.1 | | | 0.5 | 0.2 | |
| 8-Hexadecene-1,16- | | 0.2 | | | | |
| dicarboxylic acid | | | | | | |
| Vitamin E acetate | 0.2 | 0.2 | | 0.3 | 0.1 | 0.5 |
| Na₂H₂EDTA | 0.1 | 0.1 | 0.2 | 0.2 | 0.2 | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to 100.0 | to 100.0 | to 100.0 | to 100.0 | to 100.0 | to 100.0 |

### Example 9

### O/W Emulsions

| Emulsion | G | H | I | K | L | M |
|---|---|---|---|---|---|---|
| Ceteareth-20 | 1 | 1.5 | 1 | | | |
| Sorbitan stearate | | | 0.5 | | 0.5 | |
| Glyceryl stearate SE | | | | 1 | 1 | 1.5 |
| Emulgade F ® | | | | 2.5 | 2.5 | 3 |
| Cetearyl alcohol | | | | 1 | | |
| Stearyl alcohol | | | | | 1.5 | |
| Cetyl alcohol | | | 0.5 | | | 2 |
| Acrylates/C₁₀₋₃₀ alkyl acrylate | 0.2 | 0.4 | 0.3 | 0.1 | | |
| crosspolymer | | | | | | |
| Carbomer | | | | | 0.3 | |
| Xanthan gum | | | | 0.4 | | 0.4 |
| C₁₂₋₁₅ alkyl benzoate | 5 | 3 | | | 5 | |
| 2-Phenyl benzoate | | 2 | | | | |
| Butylene glycol | 5 | | | | 3 | 2 |
| dicaprylat/dicaprat | | | | | | |
| Dicaprylyl ether | | | | | 2 | |
| Diethylhexyl naphthalate | 2 | | | | | |
| Dicapryl caprate | | 2 | | | 2 | 2 |
| Cyclomethicone | 5 | | 5 | 10 | | |
| Isohexadecane | | | | 5 | | |
| Mineral oil | | 1 | | | | |
| Propylene glycol | | | 4 | | | |
| Glycerine | 5 | 7 | 3 | 5 | 6 | 8 |
| C18-38 acid triglyceride | 0.5 | | 1 | | 1 | |
| Titanium dioxide | 5 | | 3 | 2 | | |
| NeoHeliopan ® AP | | 2 | | | 1 | 1 |
| Phenylbenzimidazolesulfonic | 1 | | | 1 | 2 | 1 |
| acid | | | | | | |
| Ethylhexyl | 5 | | 4 | 4 | | |
| methoxycinnamate | | | | | | |
| Ethylhexyltriazone | | 2 | | 1 | | |
| Diethylhexylbutamido- | 1 | | | | | |
| triazane | | | | | | |
| Butylmethoxydibenzoyl- | 2.5 | | 2 | 2 | | 1 |
| methane | | | | | | |
| Bisethylhexyloxyphenol- | 2 | | | | | |
| methoxyphenyltriazine | | | | | | |
| 4-Methylbenzylidene- | 3 | | | | | |
| camphor | | | | | | |
| Parsol ® SLX | | | | | 2 | |
| (R)-2-((R)--3,4-Dihydroxy-5- | 1.0 | 2.0 | 4.0 | 0.5 | 1.5 | 3.0 |
| oxo-2,5-dihydrofuran-2-yl)-2- | | | | | | |
| hydroxyethyl 2-(4-dihexylyl- | | | | | | |
| amino-2-hydroxybenzoyl)- | | | | | | |
| benzoate | | | | | | |
| Creatinin | 0.1 | 0.01 | 0.05 | | | |
| Creatin | 0.5 | 0.2 | 0.1 | | | |
| Licorice extract/licochalcone | | | | 0.5 | | |
| Vitamin E acetate | 0.2 | | | 0.5 | 0.5 | 0.5 |
| Tapioca starch | | 3 | | | 2 | |
| Na₂H₂EDTA | 0.1 | | 0.2 | | | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to 100.0 | to 100.0 | to 100.0 | to 100.0 | to 100.0 | to 100.0 |

### Example 10

### O/W Emulsions

| Emulsion | N | O | P | Q | R | S |
|---|---|---|---|---|---|---|
| Glyceryl stearate SE | | 2 | | 2 | | |
| Glyceryl stearate | 2 | | 2 | | | |
| PEG-40 stearate | | | 2 | | 1 | |
| PEG-10 stearate | | | | 2.5 | 1 | |
| Ceteareth-20 | | | | | | 2.6 |
| Sodium cetyl phosphate | | | | | 2 | |
| Glyceryl stearate, ceteareth- | | | | | | 5.4 |
| 12, ceteareth-20, cetearyl | | | | | | |
| alcohol, cetyl palmitate | | | | | | |
| Stearic acid | 3 | 2 | | | 2 | |
| Stearyl alcohol | | 2 | 2 | | | |
| Stearyl alcohol | 0.5 | | 2 | | | |
| Cetyl alcohol | 3 | | | 2 | | |
| Acrylates/C₁₀₋₃₀ alkyl acrylate | | | 0.2 | | 0.4 | |
| crosspolymer | | | | | | |
| Carbomer | | 0.3 | | 0.3 | 0.3 | |
| Xanthan gum | | 0.3 | 0.4 | | | |
| C₁₂₋₁₅ alkyl benzoate | 5 | | | | 5 | 3 |
| 2-Phenyl benzoate | 5 | | | | | |
| Butylene glycol | | 5 | | 4 | | 3 |
| dicaprylate/dicaprate | | | | | | |
| Dicaprylyl ether | | 2 | | | 3 | |
| Diethylhexyl naphthalate | 3 | | | | | |
| Cyclomethicone | 2 | | 10 | 2 | | |
| Isohexadecane | | | | 2 | 3 | |
| Mineral oil | | | | | 3 | |
| Propanediol | | 3 | | 5 | | |
| Glycerine | 3 | 5 | 10 | 7 | 4 | 5 |
| Titanium dioxide | 2 | 4 | | | | |
| Zinc oxide | | | | | 2 | |
| Drometrizole trisiloxane | | | | | 3 | |
| Ethylhexyl | | 6 | 5 | | | |
| methoxycinnamate | | | | | | |
| Phenylbenzimidazolesulfonic | | 0.5 | 2 | | 1 | |
| acid | | | | | | |
| Homosalate | 5 | | | 7 | | |
| Butylmethoxydibenzoyl- | | 3 | | | | |
| methane | | | | | | |
| Bisethylhexyloxyphenol- | | 2 | 3 | | | |
| methoxyphenyltriazine | | | | | | |
| Octyl salicylate | | | | 5 | | |
| Octocrylene | | | | | 3 | |
| (R)-2-((R)--3,4-Dihydroxy-5- | 0.25 | 1.5 | 0.5 | 2.5 | 1.0 | 5.0 |
| oxo-2,5-dihydrofuran-2-yl)-2- | | | | | | |
| hydroxyethyl 2-(4-dihexylyl- | | | | | | |
| amino-2-hydroxybenzoyl)- | | | | | | |
| benzoate | | | | | | |
| Parsol ® SLX | 4 | | | | | 5 |
| PVP hexadecene copolymer | 0.5 | | 1 | | 0.8 | |
| Coenzyme Q 10 | 0.2 | 0.02 | | 0.3 | | |
| Vitamin E acetate | 0.2 | | 0.3 | | 0.8 | 0.5 |
| Na₂H₂EDTA | 0.1 | | | | | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to 100.0 | to 100.0 | to 100.0 | to 100.0 | to 100.0 | to 100.0 |

### Example 11

### Hydrodisperions (Lotions and Sprays)

| | A | B | C | D | E | F |
|---|---|---|---|---|---|---|
| Glyceryl stearate citrate | | 0.40 | | | | |
| Cetyl alcohol | | | | | 2.00 | |
| Sodium carbomer | | | | | 0.30 | |
| Acrylates/C₁₀₋₃₀ alkyl acrylate | 0.30 | | 0.30 | 0.40 | 0.10 | 0.10 |
| crosspolymer | | | | | | |
| Cetsareth-20 | | | 1.00 | | | |
| Xanthan gum | | | | 0.15 | | 0.50 |
| Dimethicone/vinyl- | | | | 5.00 | | 3.00 |
| dimethicone crosspolymer | | | | | | |
| UVASorb ® K2A | | | | | 3.50 | |
| Uvinul ® A Plus | 0.25 | | | 0.50 | 2.00 | 1.50 |
| Butylmethoxydibenzoyl- | 1.20 | | 3.50 | | | |
| methane | | | | | | |
| Bisethylhexyloxyphenol- | 2.00 | 2.00 | | 0.25 | | |
| methoxyphenyltriazine | | | | | | |
| Terephthalidenedicamphor- | | | | | | 0.50 |
| sulfonic acid | | | | | | |
| Disodium phenyl- | | | | | | 1.00 |
| dibenzimidazole | | | | | | |
| tetrasulfonate | | | | | | |
| Phenylbenzimidazolesulfonic | | | 2.00 | | | |
| acid | | | | | | |
| Ethylhexyl | 5.00 | | 7.00 | | 5.00 | 8.00 |
| methoxycinnamate | | | | | | |
| Diethylhexylbutamido- | | | 2.00 | 2.00 | | |
| triazone | | | | | | |
| Ethylhexyltriazone | 4.00 | 3.00 | | | 4.00 | |
| Octocrylene | | | | 10.00 | | 2.50 |
| (R)-2-((R)--3,4-Dihydroxy-5- | 0.25 | 1.5 | 0.5 | 2.5 | 1.0 | 5.0 |
| oxo-2,5-dihydrofuran-2-yl)-2- | | | | | | |
| hydroxyethyl 2-(4-dihexylyl- | | | | | | |
| amino-2-hydroxybenzoyl)- | | | | | | |
| benzoate | | | | | | |
| C₁₂₋₁₅ alkyl benzoate | 2.00 | | 2.50 | | | |
| Phenethyl benzoate | 4.00 | | | 7.50 | | 5.00 |
| C₁₈₋₃₆ triglyceride fatty acid | | | 1.00 | | | |
| Butylene glycol | | | | | 6.00 | |
| dicaprylat/dicaprate | | | | | | |
| Dicaprylyl carbonate | | 3.00 | | | | |
| Dicaprylyl ether | | 2.00 | | | | |
| Cyclomethicone | | | | 1.50 | | |
| Lanolin | | | | | 0.35 | |
| PVP hexadecene copolymer | 0.50 | | 0.50 | | 0.50 | 1.00 |
| Ethylhexyloxyglycerine | | 0.75 | | 1.00 | | 0.50 |
| Glycerine | 10.00 | 5.00 | 5.00 | | 5.00 | 15.00 |
| Butylene glycol | | 7.00 | | | | |
| Glycine soya | | | | 1.00 | | |
| Vitamin E acetate | 0.50 | 0.25 | 0 50 | 0.25 | 0.75 | 1.00 |
| α-Glycosylrutin | | | | | 0.25 | |
| Trisodium EDTA | | 1.00 | 1.00 | 0.10 | 0.20 | |
| Idopropynyl butylcarbamate | 0.20 | 0.10 | | | | 0.15 |
| Methylparaben | 0.50 | | 0.20 | | 0.15 | |
| Phenoxyethanol | 0.50 | 0.40 | 0.40 | | 1.00 | 0.60 |
| Ethanol | 3.00 | 10.00 | 4.00 | 3.50 | | 1.00 |
| Perume, dyes | q.s. | q.s. | q.s. | qs. | q.s. | q.s. |
| Water | to 100 | to 100 | to 100 | to 100 | to 100 | to 100 |
| Neutralisers (sodium | qs | qs | qs | qs | qs | qs |
| hydroxide, potassium | | | | | | |
| hydroxid) | | | | | | |

### Example 12

### Aqueous and Aqueous/Alcoholic Formulations

| | A | E | C | D | E | F |
|---|---|---|---|---|---|---|
| Ethanol | 50 | 5 | 2 | 40 | 15 | |
| Hydroxyethylcellulose | 0.5 | | | | | |
| Acrylates/C10-30 alkyl acrylate | | | | 0.3 | 0.6 | |
| crosspolymer | | | | | | |
| Cocoatnidopropylbetain | | | 0.3 | | | |
| UVASorb ® K2A | | | | | 2 | |
| Uvinul ® A Plus | 5 | | | | | |
| Butylmethoxydibenzoylmethane | 0.5 | | | 3 | | |
| Disodium phenyldibenzimidazoletetrasulfonate | | 2 | 1 | | | |
| Phenylbenzimidazolesulfonic | | 5 | 3 | | 2 | 4 |
| acid | | | | | | |
| Ethylhexyl methoxycinnamate | 10 | | | | 3 | |
| Diethylhexylbutamidotriazone | | | | 3 | | |
| Ethylhexyltriazone | | | | | 2 | |
| Octocrylene | | | | 5 | | |
| (R)-2-((R)--3,4-Dihydroxy-5-oxo- | 2.5 | 0.75 | 1.5 | 3.0 | 3.5 | 4.0 |
| 2,5-dihydrofuran-2-yl)-2-hydroxy- | | | | | | |
| ethyl 2-(4-dihexylylamino-2- | | | | | | |
| hydroxybenzoyl)benzoate | | | | | | |
| C₁₂₋₁₅ alkyl benzoate | | | | 3 | | |
| C18-36 triglyceride fatty acid | | | | 1 | | |
| Butylene glycol | 2 | | | | | |
| dicaprylate/dicaprate | | | | | | |
| C12-13 alkyl tartrate | | | | | 5 | |
| Cyclomethicone | 4 | | | 2 | | |
| Insect repellent ® 3535 | | | | 5 | | |
| Dimethicone | | | | | 3 | |
| PVP hexadecene copolymer | | 0.5 | | 1 | | 0.5 |
| Ethylhexyloxyglycerine | | 0.5 | | | | |
| Glycerine | 5 | 7 | 3 | 8 | | S |
| Butylene glycol | | | 5 | | 5 | |
| Metylpropanediol | | | | 4 | | |
| Vitamin E acetate | | 0.3 | 0.2 | 0.5 | | |
| Panthenol | 0.5 | | 0.2 | | | 0.3 |
| Creatinin | | | 0.01 | | 0.02 | |
| Creatin | | | 0.1 | | 0.2 | |
| PEG-40 hydrogenated castor | | 0.5 | 0.3 | | | 0.5 |
| oil | | | | | | |
| Trisodium EDTA | 0.3 | 0.2 | 0.2 | 0.2 | 0.2 | 0.5 |
| Preservatives | q.s. | q.s. | q.s. | q.s. | q.s. | q.s. |
| Sodium hydroxide | q.s. | q.s. | q.s. | q.s. | q.s. | q.s. |
| Perfume, dyes | q.s. | q.s. | q.s. | q.s. | q.s. | q.s. |
| Water | to 100 | to 100 | to 100 | to 100 | to 100 | to 100 |

### Example 13

### Cosmetic Foams

| Emulsion | A | B | C |
|---|---|---|---|
| Stearic acid | 2 | 2 | |
| Palmitic acid | | | 1.5 |
| Cetyl alcohol | 2.5 | 2 | |
| Stearyl alcohol | | | 3 |
| PEG-100 stearate | | | 3.5 |
| PEG-40 stearate | | 2 | |
| PEG-20 stearate | 3 | | |
| Sorbitan stearate | | 0.8 | |
| C₁₂₋₁₅ alkyl benzoate | 5 | | |
| C₁₂₋₁₃ alkyl tartrate | | | 7 |
| Butylene glycol | | 6 | |
| dicaprylate/dicaprate | | | |
| Dicaprylyl ether | | | 2 |
| Cyclomethicone | | 2 | 3 |
| Butylene glycol | 1 | | |
| Isohexadecane | 2 | | |
| Methylpropanediol | | | |
| Propylene glycol | | | 5 |
| Glycerine | 5 | 7 | |
| UVASorb ® K2A | | | 2 |
| Uvinul ® A Plus | 2 | 3 | |
| (R)-2-((R)-3,4-Dihydroxy-5-oxo-2,5- | 0.5 | 1.0 | 1.5 |
| dihydrofuran-2-yl)-2-hydroxyethyl 2-(4- | | | |
| dihexylylamino-2-hydroxybenzoyl)- | | | |
| benzoate | | | |
| Parsol SLX ® | | 3 | |
| Homosalate | | 5 | |
| Phenylbenzimidazolesulfonic acid | | 2 | 2 |
| Benzophenone-3 | 2 | | |
| Octylsalicylate | | 5 | |
| Octocrylene | 2 | | |
| Bisethylhexyloxyphenol- | | 3 | |
| methoxyphenyltriazine | | | |
| 2.2′-Methylenebis(6-(2H- | | | 8 |
| benzotriazol-2-yl)-4-(1,1,3,3- | | | |
| tetramethylbutyl)phenol) | | | |
| 2,4,6-Tris(biphenyl)-1,3,5-triazine | 5 | | 4 |
| C8-C16 alkylpolyglycoside | 1 | | |
| Vitamin E acetate | 0.6 | 0.5 | 0.2 |
| Creatin/creatinin | | | 0.5 |
| BHT | | | 0.1 |
| Na₂H₂EDTA | 0.50 | | |
| Perfume, preservatives | q.s. | q.s. | q.s. |
| Dyes, etc. | q.s. | q.s. | q.s. |
| Sodium hydroxide | q.s. | | q.s. |
| Potassium hydroxide | | q.s. | |
| Water | to 100.0 | to 100.0 | to 100.0 |

### Example 14

### Cosmetic Foams

| Emulsion | D | E | F | G |
|---|---|---|---|---|
| Stearic acid | 2 | | | |
| Palmitic acid | | | 3 | 3 |
| Cetyl alcohol | 2 | 2 | | |
| Cetylstearyl alcohol | | | 2 | 2 |
| Stearyl alcohol | | | | |
| PEG-100 stearate | | 4 | | |
| PEG-40 stearate | 2 | | | |
| PEG-20 stearate | | | 3 | 3 |
| Sorbitan stearate | 0.8 | | | |
| Tridecyl trimellitate | | 5 | | |
| C₁₂₋₁₅ alkyl benzoate | | | 3 | 3 |
| Butylene glycol | 8 | | | |
| dicaprylate/dicaprate | | | | |
| Octyldodecanol | | 2 | | |
| Cocoglyceride | | | | 2 |
| Dicaprylyl ether | | | 2 | 2 |
| Cyclomethicone | | | | |
| Dimethicone | 1 | | 2 | 2 |
| Isohexadecane | | 3 | | |
| Methylpropanediol | | 4 | | |
| Propylene glycol | | | | |
| Glycerine | 5 | | 6 | 6 |
| NeoHeliopan ® AP | | 2 | | |
| Phenylbenzimidazole- | 1 | | | 1 |
| sulfonic acid | | | | |
| (R)-2-((R)--3,4-Dihydroxy-5- | 0.75 | 1.5 | 3.0 | 6.0 |
| oxo-2,5-dihydrofuran-2-yl)-2- | | | | |
| hydroxyethyl 2-(4-dihexylyl- | | | | |
| amino-2-hydroxybenzoyl)- | | | | |
| benzoate | | | | |
| Ethylhexyl | 5 | | 4 | 4 |
| methoxycinnamate | | | | |
| Ethylhexyltriazone | | 2 | | 1 |
| Eusolex T-AVO ® | 2 | | | |
| Diethylhexylbutamido- | 1 | | | |
| triazone | | | | |
| Butylmethoxydibenzoyl- | 2.5 | | 2 | 2 |
| methane | | | | |
| Bisethylhexyloxyphenol- | 2 | | | |
| methoxyphenyltriazine | | | | |
| Vitamin E acetate | 0.2 | | 0.3 | 0.3 |
| Na₂H₂EDTA | | | | |
| Perfume, preservatives | | | | |
| Dyes, etc. | | | | |
| Sodium hydroxide | | q-s. | q.s. | |
| Triethanolamine | q.s. | | | q.s. |
| Water | to 100.0 | to 100.0 | to 100.0 | to 100.0 |

1. A method for the functionalisation of matrices, comprising using at least one ascorbic acid derivative conforming to the formula I 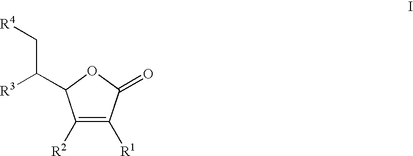
where
R¹ or R² are each, independently of one another, hydroxyl, —O-alkyl, —OC(O)-alkyl, —OPO₃M or O-glycosyl,
alkyl is C₁-C₂₀-alkyl,
M is an alkali or alkaline-earth metal cation or H,
R³ or R⁴ are each, independently of one another, hydroxyl or a radical B and
B is a substituent of the formula II 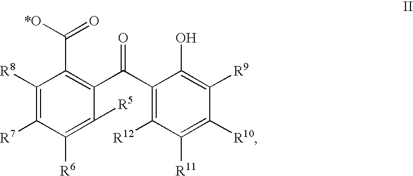
where
R⁵ to R⁹ and R¹¹ to R¹² each, independently of one another, denote H, —OH, —OA, -A, —NH₂, —NHA, —NA₂, —NH—(CH₂—CH₂—O)ₙ—H, —N[(CH₂—CH₂—Oₙ—H]₂, —[NHA₂]X, —[NA₃]X, —SO₃H, —[SO₃]X or 2H-benzotriazol-2-yl and
A is alkyl having 1 to 20 C atoms,
n is an integer from 1 to 25,
X is the counterion to the cations [NHA₂]⁺ and [NA₃]⁺ or the anion [SO₃]⁻ and
Y and Z are each, independently of one another, -ascorbyl, hydroxyl, —O-2-ethylhexyl, —O-hexyl, —OA or —NH—C(CH₃)₃ and R¹⁰ stands for A, NA′₂ or OA′, where A′ denotes branched or linear C₅-C₂₀ alkyl, with the proviso that at least one of the radicals R³ or R⁴ stands for the radical B.

2. A method according to claim 1, characterised in that the matrix is skin, hair or nails.

3. A method claim 1, characterised in that R² in formula I denotes hydroxyl.

4. A method according to claim 1, characterised in that R¹ in formula I denotes hydroxyl.

5. A method according to claim 1, characterised in that R¹⁰ in formula II denotes NA′₂.

6. Compounds of the formula I 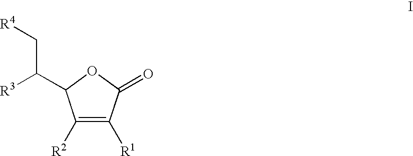
where
R¹ or R² are each, independently of one another, hydroxyl, —O-alkyl, —OC(O)-alkyl, —OPO₃M or O-glycosyl,
alkyl is C₁-C₂₀-alkyl,
M is an alkali or alkaline-earth metal cation or H,
R³ or R⁴ are each, independently of one another, hydroxyl or a radical B and
B is a substituent of the formula II 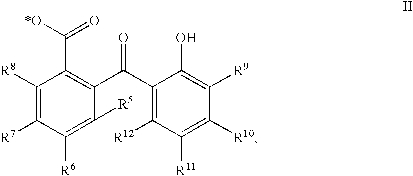
where
R⁵ to R⁹ and R¹¹ to R¹² each, independently of one another, denote H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl and
A is alkyl having 1 to 20 C atoms,
n is an integer from 1 to 25,
X is the counterion to the cations [NHA₂]⁺ and [NA₃]⁺ or the anion [SO₃]⁻ and Y and Z are each, independently of one another, -ascorbyl, hydroxyl, —O-2-ethylhexyl, —O-hexyl, —OA or —NH—C(CH₃)₃ and R¹⁰ stands for A, NA′₂ or OA′, where A′ denotes branched or linear C₅-C₂₀ alkyl, with the proviso that at least one of the radicals R³ or R⁴ stands for the radical B.

7. Compounds according to claim 6, characterised in that R² in formula I denotes hydroxyl.

8. Compounds according to claim 6, characterised in that R¹ in formula I denotes hydroxyl.

9. Compounds according to claim 6, characterised in that R⁵ to R⁹, R¹¹ and R¹² in formula II denote H.

10. Compounds according to claim 6, characterised in that R¹⁰ in formula II denotes NA′₂.

11. Process for the preparation of compounds according to claim 6, characterised in that
a) a compound of the formula III 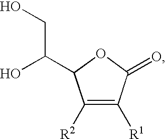
in which
R¹ or R² has a meaning described in claim 6,
is reacted directly with a compound of the formula IV
B-M IV
in which B has a meaning described in claim 6, and
M denotes an alkali metal cation or alkaline-earth metal cation or H, or
b) the hydroxyl groups of the compound of the formula III, as described above, are protected to give a compound of the formula V 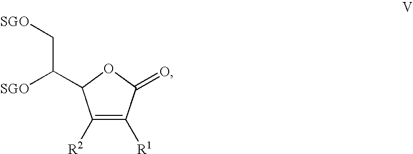
in which
R¹ or R² where has the compound of the formula I,
the radicals R¹ and/or R², if these are hydroxyl groups, are subsequently protected by a second protecting group which can be cleaved off again under different reaction conditions to the protecting group SG,
the protecting groups SG of the compounds of the formula V are cleaved off again, and the resultant compound is reacted with a compound of the formula IV
B-M IV,
where has the compound of the formula I, and M denotes an alkali metal cation or alkaline-earth metal cation or H, and the radicals R¹ and/or R² are subsequently deprotected as hydroxyl group, and these hydroxyl groups are optionally converted into another radical R¹ or R²≠OH.

12. Composition comprising at least one compound according to claim 6.

13. Composition according to claim 12, characterised in that it comprises a cosmetically or pharmacologically compatible vehicle.

14. Composition according to claim 12, characterised in that the at least one compound of the formula I is present in amounts of 0.05 to 10% by weight.

15. Composition according to claim 12, characterised in that at least one further organic UV filter is present.

16. Composition according to claim 12, characterised in that at least one inorganic UV filter is present.

17. Composition according to claim 12, characterised in that at least one further ascorbic acid derivative, preferably from the group ascorbic acid, magnesium ascorbyl phosphate or ascorbyl palmitate, is present.

18. Composition according to claim 12, characterised in that at least one antioxidant is present.

19. Composition according to claim 12, characterised in that at least one anti-ageing active compound and/or at least one anti-cellulite active compound is present.

20. Composition according to claim 12, characterised in that at least one vitamin derivative is present.

21. Composition according to claim 12, characterised in that at least one further auxiliary, selected from the group thickeners, softeners, moisturisers, surface-active agents, emulsifiers, preservatives, antifoams, perfumes, waxes, lanolin, propellants, dyes and/or pigments which colour the composition itself or the skin, is present

22. Process for the preparation of a composition according to claim 13, characterised in that at least one said compound is mixed with a vehicle and optionally with further active compounds or auxiliaries.

23. A method of skin- and/or hair-binding UV filters filtering, comprising using a compound of Formula I of claim 6.
